SEQUENCE LISTING (153 sequences)

SEQ ID NO: 1 - protein, 28 aa, Homo sapiens
SLRRSSCFGGRMDRIGAQSGLGCNSFRY

SEQ ID NO: 2 - protein, 32 aa, Homo sapiens
SPKMVQGSGCFGRKMDRISSSSGLGCKVLRRH

SEQ ID NO: 3 - protein, 22 aa, Homo sapiens
GLSKGCFGLKLDRIGSMSGLGC

SEQ ID NO: 4 - protein, 32 aa, Homo sapiens
TAPRSLRRSSCFGGRMDRIGAQSGLGCNSFRY

SEQ ID NO: 5 - protein, 38 aa, Dendroaspis
EVKYDPCFGHKIDRINHVSNLGCPSLRDPRPNAPSTSA

SEQ ID NO: 6 - protein, 26 aa, Homo sapiens
SPKMVQGSGCFGRKMDRISSSSGLGC

SEQ ID NO: 7 - protein, 15 aa, Dendroaspis
PSLRDPRPNAPSTSA

SEQ ID NO: 8 - protein, 41 aa, Dendroaspis
SPKMVQGSGCFGRKMDRISSSSGLGCPSLRDPRPNAPSTSA

SEQ ID NO: 9 - protein, 37 aa, Artificial Sequence
  synthetic peptide
GLSKGCFGLKLDRIGSMSGLGCPSLRDPRPNAPSTSA

SEQ ID NO: 10 - protein, 10 aa, Homo sapiens
TAPRSLRRSS

SEQ ID NO: 11 - protein, 17 aa, Homo sapiens
CFGLKLDRIGSMSGLGC

SEQ ID NO: 12 - protein, 5 aa, Homo sapiens
NSFRY

SEQ ID NO: 13 - protein, 32 aa, Artificial Sequence
  synthetic polypeptides
TAPRSLRRSSCFGLKLDRIGSMSGLGCNSFRY

SEQ ID NO: 14 - protein, 6 aa, Homo sapiens
SLRRSS

SEQ ID NO: 15 - protein, 17 aa, Homo sapiens
CFGRKMDRISSSSGLGC

SEQ ID NO: 16 - protein, 28 aa, Artificial Sequence
  synthetic polypeptides
SLRRSSCFGRKMDRISSSSGLGCNSFRY

SEQ ID NO: 17 - protein, 32 aa, Artificial Sequence
  synthetic polypeptides
TAPRSLRRSSCFGRKMDRISSSSGLGCNSFRY

SEQ ID NO: 18 - protein, 28 aa, Artificial Sequence
  synthetic polypeptides
SLRRSSCFGLKLDRIGSMSGLGCNSFRY

SEQ ID NO: 19 - protein, 6 aa, Homo sapiens
KVLRRH

SEQ ID NO: 20 - protein, 29 aa, Artificial Sequence
  synthetic polypeptides
SLRRSSCFGLKLDRIGSMSGLGCKVLRRH

SEQ ID NO: 21 - protein, 9 aa, Homo sapiens
SPKMVQGSG

SEQ ID NO: 22 - protein, 32 aa, Artificial Sequence
  synthetic polypeptides
SPKMVQGSGCFGLKLDRIGSMSGLGCKVLRRH

SEQ ID NO: 23 - protein, 6 aa, Dendroaspis
EVKYDP

SEQ ID NO: 24 - protein, 38 aa, Artificial Sequence
  synthetic polypeptides
EVKYDPCFGLKLDRIGSMSGLGCPSLRDPRPNAPSTSA

SEQ ID NO: 25 - protein, 33 aa, Artificial Sequence
  synthetic polypeptides
TAPRSLRRSSCFGLKLDRIGSMSGLGCKVLRRH

SEQ ID NO: 26 - protein, 5 aa, Homo sapiens
RMDRI

SEQ ID NO: 27 - protein, 33 aa, Artificial Sequence
  synthetic polypeptides
RMDRIGLSKGCFGLKLDRIGSMSGLGCKVLRRH

SEQ ID NO: 28 - protein, 22 aa, Artificial Sequence
  synthetic polypeptides
GLSKGCFGLKLDRIREASGLGC

SEQ ID NO: 29 - protein, 33 aa, Artificial Sequence
  synthetic polypeptides
RMDRIGLSKGCFGLKLDRIREASGLGCKVLRRH

SEQ ID NO: 30 - protein, 28 aa, Artificial Sequence
  synthetic polypeptides
GLSKGCFGLKLDRIREASGLGCKVLRRH

SEQ ID NO: 31 - protein, 28 aa, Artificial Sequence
  synthetic polypeptides
TLRRSSCFGGRMDRIGAQSGLGCNSFRY

SEQ ID NO: 32 - protein, 28 aa, Artificial Sequence
  synthetic polypeptides
SIRRSSCFGGRMDRIGAQSGLGCNSFRY

SEQ ID NO: 33 - protein, 28 aa, Artificial Sequence
  synthetic polypeptides
SLKRSSCFGGRMDRIGAQSGLGCNSFRY

SEQ ID NO: 34 - protein, 28 aa, Artificial Sequence
  synthetic polypeptides
SLRKSSCFGGRMDRIGAQSGLGCNSFRY

SEQ ID NO: 35 - protein, 28 aa, Artificial Sequence
  synthetic polypeptides
SLRRSSCFGGRMDRIGAQSGLGCNTFRY

SEQ ID NO: 36 - protein, 28 aa, Artificial Sequence
  synthetic polypeptides
SLRRSSCFGGRMDRIGAQSGLGCNSLRY

SEQ ID NO: 37 - protein, 28 aa, Artificial Sequence
  synthetic polypeptides
SLRRSSCFGGRMDRIGAQSGLGCNSFKY

SEQ ID NO: 38 - protein, 28 aa, Artificial Sequence
  synthetic polypeptides
SLRRSSCFGGRMDRIGAQSGLGCNSFRF

SEQ ID NO: 39 - protein, 32 aa, Artificial Sequence
  synthetic polypeptides
TPKMVQGSGCFGRKMDRISSSSGLGCKVLRRH

SEQ ID NO: 40 - protein, 32 aa, Artificial Sequence
  synthetic polypeptides
SGKMVQGSGCFGRKMDRISSSSGLGCKVLRRH

SEQ ID NO: 41 - protein, 32 aa, Artificial Sequence
  synthetic polypeptides
SPRMVQGSGCFGRKMDRISSSSGLGCKVLRRH

SEQ ID NO: 42 - protein, 32 aa, Artificial Sequence
  synthetic polypeptides
SPKLVQGSGCFGRKMDRISSSSGLGCKVLRRH

SEQ ID NO: 43 - protein, 32 aa, Artificial Sequence
  synthetic polypeptides
SPKMVQGSGCFGRKMDRISSSSGLGCKVIRRH

SEQ ID NO: 44 - protein, 32 aa, Artificial Sequence
  synthetic polypeptides
SPKMVQGSGCFGRKMDRISSSSGLGCKVLKRH

SEQ ID NO: 45 - protein, 32 aa, Artificial Sequence
  synthetic polypeptides
SPKMVQGSGCFGRKMDRISSSSGLGCKVLRKH

SEQ ID NO: 46 - protein, 32 aa, Artificial Sequence
  synthetic polypeptides
SPKMVQGSGCFGRKMDRISSSSGLGCKVLRRR

SEQ ID NO: 47 - protein, 22 aa, Artificial Sequence
  synthetic polypeptides
PLSKGCFGLKLDRIGSMSGLGC

SEQ ID NO: 48 - protein, 22 aa, Artificial Sequence
  synthetic polypeptides
GISKGCFGLKLDRIGSMSGLGC

SEQ ID NO: 49 - protein, 22 aa, Artificial Sequence
  synthetic polypeptides
GLTKGCFGLKLDRIGSMSGLGC

SEQ ID NO: 50 - protein, 22 aa, Artificial Sequence
  synthetic polypeptides
GLSRGCFGLKLDRIGSMSGLGC

SEQ ID NO: 51 - protein, 22 aa, Artificial Sequence
  synthetic polypeptides
GLSKGCFGLKLDRIGSMSPLGC

SEQ ID NO: 52 - protein, 22 aa, Artificial Sequence
  synthetic polypeptides
GLSKGCFGLKLDRIGSMSGIGC

SEQ ID NO: 53 - protein, 22 aa, Artificial Sequence
  synthetic polypeptides
GLSKGCFGLKLDRIGSMSGLPC

SEQ ID NO: 54 - protein, 22 aa, Artificial Sequence
  synthetic polypeptides
GLSKGCFGLKLDRIGSMSGLGS

SEQ ID NO: 55 - protein, 41 aa, Artificial Sequence
  synthetic polypeptides
TPKMVQGSGCFGRKMDRISSSSGLGCPSLRDPRPNAPSTSA

SEQ ID NO: 56 - protein, 41 aa, Artificial Sequence
  synthetic polypeptides
SGKMVQGSGCFGRKMDRISSSSGLGCPSLRDPRPNAPSTSA

SEQ ID NO: 57 - protein, 41 aa, Artificial Sequence
  synthetic polypeptides
SPRMVQGSGCFGRKMDRISSSSGLGCPSLRDPRPNAPSTSA

SEQ ID NO: 58 - protein, 41 aa, Artificial Sequence
  synthetic polypeptides
SPKLVQGSGCFGRKMDRISSSSGLGCPSLRDPRPNAPSTSA

SEQ ID NO: 59 - protein, 41 aa, Artificial Sequence
  synthetic polypeptides
SPKMVQGSGCFGRKMDRISSSSGLGCPSLRDPRPNAPTTSA

SEQ ID NO: 60 - protein, 41 aa, Artificial Sequence
  synthetic polypeptides
SPKMVQGSGCFGRKMDRISSSSGLGCPSLRDPRPNAPSSSA

SEQ ID NO: 61 - protein, 41 aa, Artificial Sequence
  synthetic polypeptides
SPKMVQGSGCFGRKMDRISSSSGLGCPSLRDPRPNAPSTTA

SEQ ID NO: 62 - protein, 41 aa, Artificial Sequence
  synthetic polypeptides
SPKMVQGSGCFGRKMDRISSSSGLGCPSLRDPRPNAPSTSV

SEQ ID NO: 63 - protein, 37 aa, Artificial Sequence
  synthetic polypeptides
PLSKGCFGLKLDRIGSMSGLGCPSLRDPRPNAPSTSA

SEQ ID NO: 64 - protein, 37 aa, Artificial Sequence
  synthetic polypeptides
GISKGCFGLKLDRIGSMSGLGCPSLRDPRPNAPSTSA

SEQ ID NO: 65 - protein, 37 aa, Artificial Sequence
  synthetic polypeptides
GLTKGCFGLKLDRIGSMSGLGCPSLRDPRPNAPSTSA

SEQ ID NO: 66 - protein, 37 aa, Artificial Sequence
  synthetic polypeptides
GLSRGCFGLKLDRIGSMSGLGCPSLRDPRPNAPSTSA

SEQ ID NO: 67 - protein, 37 aa, Artificial Sequence
  synthetic polypeptides
GLSKGCFGLKLDRIGSMSGLGCPSLRDPRPNAPTTSA

SEQ ID NO: 68 - protein, 37 aa, Artificial Sequence
  synthetic polypeptides
GLSKGCFGLKLDRIGSMSGLGCPSLRDPRPNAPSSSA

SEQ ID NO: 69 - protein, 37 aa, Artificial Sequence
  synthetic polypeptides
GLSKGCFGLKLDRIGSMSGLGCPSLRDPRPNAPSTTA

SEQ ID NO: 70 - protein, 37 aa, Artificial Sequence
  synthetic polypeptides
GLSKGCFGLKLDRIGSMSGLGCPSLRDPRPNAPSTSV

SEQ ID NO: 71 - protein, 32 aa, Artificial Sequence
  synthetic polypeptides
SAPRSLRRSSCFGLKLDRIGSMSGLGCNSFRY

SEQ ID NO: 72 - protein, 32 aa, Artificial Sequence
  synthetic polypeptides
TVPRSLRRSSCFGLKLDRIGSMSGLGCNSFRY

SEQ ID NO: 73 - protein, 32 aa, Artificial Sequence; identical to SEQ ID NO: 72
  synthetic polypeptides

SEQ ID NO: 74 - protein, 32 aa, Artificial Sequence
  synthetic polypeptides
TAPKSLRRSSCFGLKLDRIGSMSGLGCNSFRY

SEQ ID NO: 75 - protein, 32 aa, Artificial Sequence
  synthetic polypeptides
TAPRSLRRSSCFGLKLDRIGSMSGLGCNTFRY

SEQ ID NO: 76 - protein, 32 aa, Artificial Sequence
  synthetic polypeptides
TAPRSLRRSSCFGLKLDRIGSMSGLGCNSLRY

SEQ ID NO: 77 - protein, 32 aa, Artificial Sequence
  synthetic polypeptides
TAPRSLRRSSCFGLKLDRIGSMSGLGCNSFKY

SEQ ID NO: 78 - protein, 32 aa, Artificial Sequence
  synthetic polypeptides
TAPRSLRRSSCFGLKLDRIGSMSGLGCNSFRF

SEQ ID NO: 79 - protein, 28 aa, Artificial Sequence
  synthetic polypeptides
TLRRSSCFGRKMDRISSSSGLGCNSFRY

SEQ ID NO: 80 - protein, 28 aa, Artificial Sequence
  synthetic polypeptides
SIRRSSCFGRKMDRISSSSGLGCNSFRY

SEQ ID NO: 81 - protein, 28 aa, Artificial Sequence
  synthetic polypeptides
SLKRSSCFGRKMDRISSSSGLGCNSFRY

SEQ ID NO: 82 - protein, 28 aa, Artificial Sequence
  synthetic polypeptides
SLRKSSCFGRKMDRISSSSGLGCNSFRY

SEQ ID NO: 83 - protein, 28 aa, Artificial Sequence
  synthetic polypeptides
SLRRSSCFGRKMDRISSSSGLGCNTFRY

SEQ ID NO: 84 - protein, 28 aa, Artificial Sequence
  synthetic polypeptides
SLRRSSCFGRKMDRISSSSGLGCNSLRY

SEQ ID NO: 85 - protein, 28 aa, Artificial Sequence
  synthetic polypeptides
SLRRSSCFGRKMDRISSSSGLGCNSFKY

SEQ ID NO: 86 - protein, 28 aa, Artificial Sequence
  synthetic polypeptides
SLRRSSCFGRKMDRISSSSGLGCNSFRF

SEQ ID NO: 87 - protein, 32 aa, Artificial Sequence
  synthetic polypeptides
SAPRSLRRSSCFGRKMDRISSSSGLGCNSFRY

SEQ ID NO: 88 - protein, 32 aa, Artificial Sequence
  synthetic polypeptides
TVPRSLRRSSCFGRKMDRISSSSGLGCNSFRY

SEQ ID NO: 89 - protein, 32 aa, Artificial Sequence
  synthetic polypeptides
TAGRSLRRSSCFGRKMDRISSSSGLGCNSFRY

SEQ ID NO: 90 - protein, 32 aa, Artificial Sequence
  synthetic polypeptides
TAPKSLRRSSCFGRKMDRISSSSGLGCNSFRY

SEQ ID NO: 91 - protein, 32 aa, Artificial Sequence
  synthetic polypeptides
TAPRSLRRSSCFGRKMDRISSSSGLGCNTFRY

SEQ ID NO: 92 - protein, 32 aa, Artificial Sequence
  synthetic polypeptides
TAPRSLRRSSCFGRKMDRISSSSGLGCNSLRY

SEQ ID NO: 93 - protein, 32 aa, Artificial Sequence
  synthetic polypeptides
TAPRSLRRSSCFGRKMDRISSSSGLGCNSFKY

SEQ ID NO: 94 - protein, 32 aa, Artificial Sequence
  synthetic polypeptides
TAPRSLRRSSCFGRKMDRISSSSGLGCNSFRF

SEQ ID NO: 95 - protein, 29 aa, Artificial Sequence
  synthetic polypeptides
TLRRSSCFGLKLDRIGSMSGLGCKVLRRH

SEQ ID NO: 96 - protein, 29 aa, Artificial Sequence
  synthetic polypeptides
SIRRSSCFGLKLDRIGSMSGLGCKVLRRH

SEQ ID NO: 97 - protein, 29 aa, Artificial Sequence
  synthetic polypeptides
SLKRSSCFGLKLDRIGSMSGLGCKVLRRH

SEQ ID NO: 98 - protein, 29 aa, Artificial Sequence
  synthetic polypeptides
SLRKSSCFGLKLDRIGSMSGLGCKVLRRH

SEQ ID NO: 99 - protein, 29 aa, Artificial Sequence
  synthetic polypeptides
SLRRSSCFGLKLDRIGSMSGLGCKVIRRH

SEQ ID NO: 100 - protein, 29 aa, Artificial Sequence
  synthetic polypeptides
SLRRSSCFGLKLDRIGSMSGLGCKVLKRH

SEQ ID NO: 101 - protein, 29 aa, Artificial Sequence
  synthetic polypeptides
SLRRSSCFGLKLDRIGSMSGLGCKVLRKH

SEQ ID NO: 102 - protein, 29 aa, Artificial Sequence
  synthetic polypeptides
SLRRSSCFGLKLDRIGSMSGLGCKVLRRR

SEQ ID NO: 103 - protein, 32 aa, Artificial Sequence
  synthetic polypeptides
TPKMVQGSGCFGLKLDRIGSMSGLGCKVLRRH

SEQ ID NO: 104 - protein, 32 aa, Artificial Sequence
  synthetic polypeptides
SGKMVQGSGCFGLKLDRIGSMSGLGCKVLRRH

SEQ ID NO: 105 - protein, 32 aa, Artificial Sequence
  synthetic polypeptides
SPRMVQGSGCFGLKLDRIGSMSGLGCKVLRRH

SEQ ID NO: 106 - protein, 32 aa, Artificial Sequence
  synthetic polypeptides
SPKLVQGSGCFGLKLDRIGSMSGLGCKVLRRH

SEQ ID NO: 107 - protein, 32 aa, Artificial Sequence
  synthetic polypeptides
SPKMVQGSGCFGLKLDRIGSMSGLGCKVIRRH

SEQ ID NO: 108 - protein, 32 aa, Artificial Sequence
  synthetic polypeptides
SPKMVQGSGCFGLKLDRIGSMSGLGCKVLKRH

SEQ ID NO: 109 - protein, 32 aa, Artificial Sequence
  synthetic polypeptides
SPKMVQGSGCFGLKLDRIGSMSGLGCKVLRKH

SEQ ID NO: 110 - protein, 32 aa, Artificial Sequence
  synthetic polypeptides
SPKMVQGSGCFGLKLDRIGSMSGLGCKVLRRR

SEQ ID NO: 111 - protein, 38 aa, Artificial Sequence
  synthetic polypeptides
DVKYDPCFGLKLDRIGSMSGLGCPSLRDPRPNAPSTSA

SEQ ID NO: 112 - protein, 38 aa, Artificial Sequence
  synthetic polypeptides
ELKYDPCFGLKLDRIGSMSGLGCPSLRDPRPNAPSTSA

SEQ ID NO: 113 - protein, 38 aa, Artificial Sequence
  synthetic polypeptides
EVRYDPCFGLKLDRIGSMSGLGCPSLRDPRPNAPSTSA

SEQ ID NO: 114 - protein, 38 aa, Artificial Sequence
  synthetic polypeptides
EVKFDPCFGLKLDRIGSMSGLGCPSLRDPRPNAPSTSA

SEQ ID NO: 115 - protein, 38 aa, Artificial Sequence
  synthetic polypeptides
EVKYDPCFGLKLDRIGSMSGLGCPSLRDPRPNAPTTSA

SEQ ID NO: 116 - protein, 38 aa, Artificial Sequence
  synthetic polypeptides
EVKYDPCFGLKLDRIGSMSGLGCPSLRDPRPNAPSSSA

SEQ ID NO: 117 - protein, 38 aa, Artificial Sequence
  synthetic polypeptides
EVKYDPCFGLKLDRIGSMSGLGCPSLRDPRPNAPSTTA

SEQ ID NO: 118 - protein, 38 aa, Artificial Sequence
  synthetic polypeptides
EVKYDPCFGLKLDRIGSMSGLGCPSLRDPRPNAPSTSV

SEQ ID NO: 119 - protein, 33 aa, Artificial Sequence
  synthetic polypeptides
SAPRSLRRSSCFGLKLDRIGSMSGLGCKVLRRH

SEQ ID NO: 120 - protein, 33 aa, Artificial Sequence
  synthetic polypeptides
TVPRSLRRSSCFGLKLDRIGSMSGLGCKVLRRH

SEQ ID NO: 121 - protein, 33 aa, Artificial Sequence
  synthetic polypeptides
TAGRSLRRSSCFGLKLDRIGSMSGLGCKVLRRH

SEQ ID NO: 122 - protein, 33 aa, Artificial Sequence
  synthetic polypeptides
TAPKSLRRSSCFGLKLDRIGSMSGLGCKVLRRH

SEQ ID NO: 123 - protein, 33 aa, Artificial Sequence
  synthetic polypeptides
TAPRSLRRSSCFGLKLDRIGSMSGLGCKVIRRH

SEQ ID NO: 124 - protein, 33 aa, Artificial Sequence
  synthetic polypeptides
TAPRSLRRSSCFGLKLDRIGSMSGLGCKVLKRH

SEQ ID NO: 125 - protein, 33 aa, Artificial Sequence
  synthetic polypeptides
TAPRSLRRSSCFGLKLDRIGSMSGLGCKVLRKH

SEQ ID NO: 126 - protein, 33 aa, Artificial Sequence
  synthetic polypeptides
TAPRSLRRSSCFGLKLDRIGSMSGLGCKVLRRR

SEQ ID NO: 127 - protein, 33 aa, Artificial Sequence
  synthetic polypeptides
KMDRIGLSKGCFGLKLDRIGSMSGLGCKVLRRH

SEQ ID NO: 128 - protein, 33 aa, Artificial Sequence
  synthetic polypeptides
RLDRIGLSKGCFGLKLDRIGSMSGLGCKVLRRH

SEQ ID NO: 129 - protein, 33 aa, Artificial Sequence
  synthetic polypeptides
RMERIGLSKGCFGLKLDRIGSMSGLGCKVLRRH

SEQ ID NO: 130 - protein, 33 aa, Artificial Sequence
  synthetic polypeptides
RMDKIGLSKGCFGLKLDRIGSMSGLGCKVLRRH

SEQ ID NO: 131 - protein, 33 aa, Artificial Sequence
  synthetic polypeptides
RMDRIGLSKGCFGLKLDRIGSMSGLGCKVIRRH

SEQ ID NO: 132 - protein, 33 aa, Artificial Sequence
  synthetic polypeptides
RMDRIGLSKGCFGLKLDRIGSMSGLGCKVLKRH

SEQ ID NO: 133 - protein, 33 aa, Artificial Sequence
  synthetic polypeptides
RMDRIGLSKGCFGLKLDRIGSMSGLGCKVLRKH

SEQ ID NO: 134 - protein, 33 aa, Artificial Sequence
  synthetic polypeptides
RMDRIGLSKGCFGLKLDRIGSMSGLGCKVLRRR

SEQ ID NO: 135 - protein, 33 aa, Artificial Sequence
  synthetic polypeptides
KMDRIGLSKGCFGLKLDRIREASGLGCKVLRRH

SEQ ID NO: 136 - protein, 33 aa, Artificial Sequence
  synthetic polypeptides
RLDRIGLSKGCFGLKLDRIREASGLGCKVLRRH

SEQ ID NO: 137 - protein, 33 aa, Artificial Sequence
  synthetic polypeptides
RMERIGLSKGCFGLKLDRIREASGLGCKVLRRH

SEQ ID NO: 138 - protein, 33 aa, Artificial Sequence
  synthetic polypeptides
RMDKIGLSKGCFGLKLDRIREASGLGCKVLRRH

SEQ ID NO: 139 - protein, 33 aa, Artificial Sequence
  synthetic polypeptides
RMDRIGLSKGCFGLKLDRIREASGLGCKVIRRH

SEQ ID NO: 140 - protein, 33 aa, Artificial Sequence
  synthetic polypeptides
RMDRIGLSKGCFGLKLDRIREASGLGCKVLKRH

SEQ ID NO: 141 - protein, 33 aa, Artificial Sequence
  synthetic polypeptides
RMDRIGLSKGCFGLKLDRIREASGLGCKVLRKH

SEQ ID NO: 142 - protein, 33 aa, Artificial Sequence
  synthetic polypeptides
RMDRIGLSKGCFGLKLDRIREASGLGCKVLRRR

SEQ ID NO: 143 - protein, 28 aa, Artificial Sequence
  synthetic polypeptides
PLSKGCFGLKLDRIREASGLGCKVLRRH

SEQ ID NO: 144 - protein, 28 aa, Artificial Sequence
  synthetic polypeptides
GISKGCFGLKLDRIREASGLGCKVLRRH

SEQ ID NO: 145 - protein, 28 aa, Artificial Sequence
  synthetic polypeptides
GLTKGCFGLKLDRIREASGLGCKVLRRH

SEQ ID NO: 146 - protein, 28 aa, Artificial Sequence
  synthetic polypeptides
GLSRGCFGLKLDRIREASGLGCKVLRRH

SEQ ID NO: 147 - protein, 28 aa, Artificial Sequence
  synthetic polypeptides
GLSKGCFGLKLDRIREASGLGCKVIRRH

SEQ ID NO: 148 - protein, 28 aa, Artificial Sequence
  synthetic polypeptides
GLSKGCFGLKLDRIREASGLGCKVLKRH

SEQ ID NO: 149 - protein, 28 aa, Artificial Sequence
  synthetic polypeptides
GLSKGCFGLKLDRIREASGLGCKVLRKH

SEQ ID NO: 150 - protein, 28 aa, Artificial Sequence
  synthetic polypeptides
GLSKGCFGLKLDRIREASGLGCKVLRRR

SEQ ID NO: 151 - protein, 151 aa, Homo sapiens
MSSFSTTTVSFLLLLAFQLLGQTRANPMYNAVSNADLMDFKNLLDHLEEKMPLEDEVVPPQVLSEPNEEAGAALSPLPEVPPWTGEVSPAQRDGGALGRGPWDSSDRSALLKSKLRALLTAPRSLRRSSCFGGRMDRIGAQSGLGCNSFRY

SEQ ID NO: 152 - protein, 163 aa, Homo sapiens
MSSFSTTTVSFLLLLAFQLLGQTRANPMYNAVSNADLMDFKNLLDHLEEKMPLEDEVVPPQVLSEPNEEAGAALSPLPEVPPWTGEVSPAQRDGGALGRGPWDSSDRSALLKSKLRALLTAPRSLRRSSCFGGRMDRIGAQSGLGCNSFRYRITAREDKQGWA

SEQ ID NO: 153 - DNA, 493 bp, Artificial Sequence
  synthetic oligonucleotides
atgagctccttttccactactactgtgtcctttctgctgctgctggccttccagctgctgggccaaacccgggctaacccaatgtacaacgccgtcagcaatgctgatctgatggattttaagaatctgctggatcacctggaggaaaagatgcctctggaagacgaagtggtccctcctcaagtcctgtccgaacctaacgaggaagccggccgctgccctgagccctctccctgaagtcccaccctggacaggggaggtcagccccgcacagagggacggaggggcactgggaagaggcccatgggatagctccgaccggagcgctctgctgaaaagcaaactgagggctctgctgaccgcccctagaagcctcaggagaagctcctgcttcggcggaaggatggaccggattggggcacaaagcggcctgggatgtaactccttccggtataggattacagctagagaagataaacagggctgggcttaa

(19) United States
(12) Patent Application Publication
(10) Pub. No.: US 2017/0333530 A1
(43) Pub. Date: Nov. 23, 2017

(54) METHODS AND MATERIALS FOR REDUCING CYSTS AND KIDNEY WEIGHT IN MAMMALS WITH POLYCYSTIC KIDNEY DISEASE

(71) Applicant: Mayo Foundation for Medical Education and Research, Rochester, MN (US)

(72) Inventors: Yasuhiro Ikeda, Rochester, MN (US); Sara J. Holditch, Rochester, MN (US); Alessandro Cataliotti, Rochester, MN (US); Vicente E. Torres, Rochester, MN (US)

(73) Assignee: Mayo Foundation for Medical Education and Research, Rochester, MN (US)

(21) Appl. No.: 15/525,344

(22) PCT Filed: Nov. 5, 2015
(86) PCT No.: PCT/US2015/059208

§ 371 Date: May 9, 2017

Related U.S. Application Data
(60) Provisional application No. 62/077,803, filed on Nov. 10, 2014.

(51) Int. Cl. A61K 38/22

(52) U.S. Cl. CPC A61K 38/22

(57) ABSTRACT

This document provides methods and materials for using natriuretic polypeptides to reduce the generation of kidney cysts, to reduce the number of kidney cysts, to reduce the size of kidney cysts, and/or to reduce the weight of a mammal's kidneys in mammals with polycystic kidney disease. For example, methods and materials for using natriuretic polypeptides (e.g., BNP) and/or nucleic acid encoding natriuretic polypeptides to reduce kidney cystogenesis and to reduce kidney organ to body weight ratios in mammals with mammals with polycystic kidney disease (e.g., autosomal recessive polycystic kidney disease) are provided.

### CROSS REFERENCE TO RELATED APPLICATIONS

This application claims the benefit of U.S. Provisional Ser. No. 62/077,803, filed Nov. 10, 2014. This disclosure of the prior application is considered part of (and is incorporated by reference in) the disclosure of this application.

### STATEMENT AS TO FEDERALLY SPONSORED RESEARCH

This invention was made with government support under DK090728 awarded by the National Institutes of Health. The government has certain rights in the invention.

### TECHNICAL FIELD

This document relates to the use of natriuretic polypeptides to reduce the generation of kidney cysts, to reduce the number of kidney cysts, to reduce the size of kidney cysts, and/or to reduce the weight of a mammal's kidneys in mammals with polycystic kidney disease. For example, this document provides methods and materials related to the use of natriuretic polypeptides (e.g., a B-type natriuretic polypeptide (BNP)) and/or nucleic acid encoding natriuretic polypeptides to reduce kidney cystogenesis and to reduce kidney organ to body weight ratios in mammals with mammals with polycystic kidney disease (e.g., autosomal recessive polycystic kidney disease).

### BACKGROUND INFORMATION

Natriuretic polypeptides (NPs) are polypeptides that can cause natriuresis (increased sodium excretion in the urine). Such polypeptides can be produced by brain, heart, kidney, and/or vascular tissue. The natriuretic peptide family in humans includes the cardiac hormones atrial natriuretic peptide (ANP), BNP, C-type natriuretic peptide (CNP), and urodilatin (URO). Natriuretic polypeptides function via well-characterized guanylyl cyclase receptors (i.e., NPR-A for ANP, BNP, and URO; and NPR-B for CNP) and the second messenger cyclic 3′5′ guanosine monophosphate (cGMP) (Kuhn, Circ. Res., 93:700-709 (2003); Tawaragi et al., Biochem. Biophys. Res. Commun., 175:645-651 (1991); and Komatsu et al., Endocrinol., 129:1104-1106 (1991)).

Polycystic kidney disease is characterized by the presence of massively enlarged fluid filled cysts in the renal tubules and/or collecting ducts. Progressive enlargement of these cysts can compromise the normal renal parenchyma, eventually leading to renal failure. Autosomal recessive polycystic kidney disease patients are likely to present with hypertension and hepatic and renal manifestations including cysts, fibrosis, and enlargement, ultimately leading to end stage renal failure, with 50% of children progressing to end stage renal failure within the first decade of life.

### SUMMARY

This document provides methods and materials for using natriuretic polypeptides to reduce the generation of kidney cysts, to reduce the number of kidney cysts, to reduce the size of kidney cysts, and/or to reduce the weight of a mammal's kidneys in mammals with polycystic kidney disease. For example, this document provides methods and materials for using natriuretic polypeptides (e.g., BNP) and/or nucleic acid encoding natriuretic polypeptides to reduce kidney cystogenesis and to reduce kidney organ to body weight ratios in mammals with mammals with polycystic kidney disease (e.g., autosomal recessive polycystic kidney disease).

As described herein, exposing mammals with polycystic kidney disease to a natriuretic polypeptide for greater than two months (e.g., greater than three, four, five, six, or more months) can result in a reduction in the number of kidney cysts being generated, a reduction in the number of kidney cysts present, a reduction in the size of kidney cysts, and/or a reduction in the weight of the mammal's kidneys. In some cases, nucleic acid vectors (e.g., AAV9 vectors) designed to have a nucleic acid sequence that encodes a natriuretic polypeptide such as ANP, BNP, CNP, or a chimeric natriuretic polypeptide (e.g., CDNP; see, e.g., WO 01/44284) can be administered to a mammal identified as being in need of a reduction in the number of kidney cysts being generated, a reduction in the number of kidney cysts present, a reduction in the size of kidney cysts, and/or a reduction in the weight of the mammal's kidneys. For example, a human previously identified as having polycystic kidney disease can be identified as being in need of a reduction in the number of kidney cysts being generated, a reduction in the number of kidney cysts present, a reduction in the size of kidney cysts, and/or a reduction in the weight of the mammal's kidneys. Once identified, that human can be treated with a natriuretic polypeptide or nucleic acid designed to express a natriuretic polypeptide for greater than two months (e.g., greater than three, four, five, six, or more months) to reduce in the number of kidney cysts being generated, to reduce the number of kidney cysts present, to reduce the size of kidney cysts, and/or to reduce the weight of the human's kidneys. Having the ability to deliver natriuretic polypeptides to mammals with polycystic kidney disease to reduce in the number of kidney cysts being generated, to reduce the number of kidney cysts present, to reduce the size of kidney cysts, and/or to reduce the weight of the human's kidneys as described herein can allow patients and clinicians to treat polycystic kidney diseases in an efficient and effective manner directed at kidney cysts and kidney weight.

As also described herein, polycystic kidney disease evolves and induces renal damage and failure. Natriuretic polypeptides, in addition to having favorable direct effects described herein on kidney cysts and the weight of kidneys, can induce an improvement of renal function and reduce tissue damage (e.g., proteinuria). Thus, natriuretic polypeptides can be used to improve kidney function and structure and to prevent and to regress renal impairment (e.g., reduced function, organ tissue damage) associated with polycystic kidney disease. As a consequence of renal impairment, cardiac function also can become impaired in mammals with PKD. The methods and materials provided herein, however, also can be used to improve cardiac function and structure in PKD.

In general, one aspect of this document features a method for treating a mammal diagnosed with polycystic kidney disease. The method comprises, or consists essentially of, (a) identifying the mammal as being in need of a reduction in kidney cysts, and (b) administering a natriuretic polypeptide or a nucleic acid encoding a natriuretic polypeptide to the mammal, wherein the number or size of kidney cysts within the mammal are reduced. The mammal can be a cat, dog, or human. The natriuretic polypeptide can be BNP. The method can comprise administering a natriuretic polypeptide to the mammal at least three times a week for at least three months. The method can comprise administering the nucleic acid to the mammal. The nucleic acid can be a viral vector. The nucleic acid can be an AAV9 or AAV2 viral vector.

In another aspect, this document features a method for treating a mammal diagnosed with polycystic kidney disease. The method comprises, or consists essentially of, (a) identifying the mammal as being in need of a reduction in kidney weight, and (b) administering a natriuretic polypeptide or a nucleic acid encoding a natriuretic polypeptide to the mammal, wherein the weight of a kidney within the mammal is reduced. The mammal can be a cat, dog, or human. The natriuretic polypeptide can be BNP. The method can comprise administering a natriuretic polypeptide to the mammal at least three times a week for at least three months. The method can comprise administering the nucleic acid to the mammal. The nucleic acid can be a viral vector. The nucleic acid can be an AAV9 or AAV2 viral vector.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic of the amino acid sequences and structures of ANP (SEQ ID NO:1), BNP (SEQ ID NO:2), CNP (SEQ ID NO:3), and DNP (SEQ ID NO:5).

FIG. 2. Low dose AAV-BNP transduction reduced kidney size in PKD rats. Kidney weight per body weight ratio was significantly reduced in low dose AAV9-BNP treated PKD rats 3 months post injection. Renotropic AAV2-BNP administration further reduced the kidney per body weight ratio. *p<0.05.

FIG. 3. Long-term BNP transduction reduced cystogenesis in PKD rats. Cyst index was measured using the H&E stained images of kidney sections of PKD rats at 3 months after low dose AAV9-BNP vector transduction. *p<0.05.

FIG. 4. High dose AAV9-BNP treatment reduced kidney size and liver cysts in PKD rats. Kidney weight to body weight ratio and liver cysts (lower panels) were substantially contracted in high dose PKD-treated rats (n=4), compared with untreated controls (n=8), 4.5 months after vector administration.

FIG. 5A. Low dose AAV9-BNP treatment reduced renal injury and improved renal function in PKD rats. Sustained BNP treatment for 3 months significantly reduced proteinuria, diminished excretion of KIM1, and improved creatinine clearance. *p<0.05.

FIG. 5B. Cyclic nucleotides level with AAV9-BNP treatment, compared to PCK rat littermate controls; excreted urine cGMP level (Left), and renal tissue isolated cAMP (Right).

FIG. 6. Sustained BNP transduction protected glomerular injury in PKD rats. H&E staining of kidney sections revealed a significant reduction in glomerular injury (sclerosis and basal membrane thickening) 3 months after low dose AAV9-BNP treatment in PKD rats. BNP therapy also reduced the expression of DESMIN in glomeruli (right panel). *p<0.05.

FIG. 7. Decreased fibrosis markers in renal tissue in BNP-treated PCK at 3 months. The levels of fibronectin-1 (Fn1) and collagen type 1 alpha 1 (Col1a1) transcripts were significantly decreased in renal tissues. *p<0.05.

FIG. 8. BNP-treated females had improved cardiac function compared to untreated littermate controls at 3 months. Treated PCK rats had preserved cardiac function (ejection fraction; Efteich), and cardiac contractility (fraction shortening; % FS). *p<0.05.

FIG. 9 contains amino acid sequences for ANP, pre-pro-ANP, mANP, and pre-pro-mANP as well as a codon optimized nucleic acid sequence that encodes pre-pro-mANP.

FIG. 10. RT-PCR quantification of Collagen type 1a1 (Col1a1), Fibronectin-1 (Fn1), Transforming growth factor-β (TGFβ), Tissue inhibitor metalloprotease-1 (Timp1), and desmin transcripts, GAPDH corrected, relative to control PCK transcripts. PCK (n=8) and AAV9-BNP (n=6). Total hepatic RNA (1 μg) analyzed.

FIG. 11. In vitro studies of relative proliferation by normal (NRC) and cystic (PCK) rat cholangiocytes derived from SD and PCK, with and without 270 nmol rat-BNP peptide supplement.

FIG. 12. Patient derived non-cystic (Normal HRE, left), and cystic renal epithelial cells (ADPKD HRE, right) were transduced with lentiviral vectors expressing control Green Fluorescent Protein (Lenti-GFP) or codon optimized human BNP (Lenti-Hu-BNP) and plated for 48 hours. Proliferation rates relative to Lenti-GFP were presented. Data represents a minimum mean of 3 individual assays, with a minimum of 20 replicates each. *P<0.05, **P<0.01, and ***P<0.001 vs Lenti-GFP by t test. Data represent the mean±SEM.

### DETAILED DESCRIPTION

This document provides methods and materials for using natriuretic polypeptides as well as nucleic acid vectors designed to express natriuretic polypeptides to reduce the generation of kidney cysts, to reduce the number of kidney cysts, to reduce the size of kidney cysts, and/or to reduce the weight of a mammal's kidneys in mammals with polycystic kidney disease.

As used herein, the term “natriuretic polypeptide” or “NP” includes native (naturally occurring, wild type) NPs (e.g., ANP, BNP, CNP, and URO, as well as Dendroaspis natriuretic peptide (DNP)), M-atrial natriuretic peptide (M-ANP; McKie et al., Curr. Hypertens. Rep., 14(1):62-9 (2012)), and chimeric NPs such as CDNP, CUNP (CNP-urodilatin), CBNP (CNP and BNP), CANP (CNP and ANP) that can combine NPR-B and NPR-A agonistic activities. The amino acid sequences for endogenous human mature NPs can be as follows:

ANP:
(SEQ ID NO: 1)
SLRRSSCFGGRMDRIGAQSGLGCNSFRY
BNP:
(SEQ ID NO: 2)
SPKMVQGSGCFGRKMDRISSSSGLGCKVLRRH
CNP:
(SEQ ID NO: 3)
GLSKGCFGLKLDRIGSMSGLGC
URO:
(SEQ ID NO: 4)
TAPRSLRRSSCFGGRMDRIGAQSGLGCNSFRY

In addition, the native Dendroaspis amino acid sequence for DNP can be EVKYDPCFGHKIDRINHVSNLGCPSLRDPRPNAPSTSA (SEQ ID NO:5).

Chimeric NPs can include amino acid sequences from two or more individual NPs. In some cases, for example, a chimeric polypeptide can include amino acid sequences from ANP and CNP; BNP and CNP; ANP, BNP, and CNP; CNP and URO; CNP and DNP; or CNP, URO, and BNP. In some cases, a chimeric NP can include a ring structure and cysteine bond (e.g., the ring structure and cysteine bond of ANP, BNP, CNP, or DNP) in combination with one or more amino acid segments from another NP. In some cases, a chimeric BD-NP can include the N-terminal 26 amino acids of human BNP (SPKMVQGSGCFGRKMDRISSSSGLGC; SEQ ID NO:6) and the C-terminal 15 amino acids of DNP (PSLRDPRPNAPSTSA; SEQ ID NO:7), and can have the amino acid sequence SPKMVQGSGCFGRKMDRISSSSGLGCPSLRDPRPNAPSTSA (SEQ ID NO:8).

In some embodiments, a chimeric CD-NP can include the amino acid sequence of human CNP (GLSKGCFGLKLDRIGSMSGLGC; SEQ ID NO:3) and the C-terminal 15 amino acids of DNP (PSLRDPRPNAPSTSA; SEQ ID NO:7), and can have the amino acid sequence GLSKGCFGLKLDRIGSMSGLGCPSLRDPRPNAPSTSA (SEQ ID NO:9).

In some embodiments, a chimeric CU-NP can include the N-terminal ten amino acids of human URO (TAPRSLRRSS; SEQ ID NO:10), the 17 amino acid ring structure and disulfide bond of human CNP (CFGLKLDRIGSMSGLGC; SEQ ID NO:11), and the C-terminal five amino acids of human URO (NSFRY; SEQ ID NO:12), and can have the amino acid sequence TAPRSLRRSSCFGLKLDRIGSMSGLGCNSFRY (SEQ ID NO:13).

In some embodiments, a chimeric BAA-NP can include the N-terminal six amino acids of human ANP (SLRRSS; SEQ ID NO:14), the 17 amino acid ring structure and disulfide bond of human BNP (CFGRKMDRISSSSGLGC; SEQ ID NO:15), and the C-terminal five amino acids of human ANP (NSFRY; SEQ ID NO:12), and can have the amino acid sequence SLRRSSCFGRKMDRISSSSGLGCNSFRY (SEQ ID NO:16).

In some embodiments, a chimeric BUA-NP can include the N-terminal 10 amino acids of human URO (TAPRSLRRSS; SEQ ID NO:10), the 17 amino acid ring structure and disulfide bond of human BNP (CFGRKMDRISSSSGLGC; SEQ ID NO:15), and the C-terminal 5 amino acids of human ANP (NSFRY; SEQ ID NO:12), and can have the amino acid sequence TAPRSLRRSSCFGRKMDRISSSSGLGCNSFRY (SEQ ID NO:17).

In some embodiments, a chimeric CAA-NP can include the N-terminal 6 amino acids of human ANP (SLRRSS; SEQ ID NO:14), the 17 amino acid ring structure and disulfide bond of human CNP (CFGLKLDRIGSMSGLGC; SEQ ID NO:11), and the C-terminal 5 amino acids of human ANP (NSFRY; SEQ ID NO:12), and can have the amino acid sequence SLRRSSCFGLKLDRIGSMSGLGCNSFRY (SEQ ID NO:18).

As another example, in some embodiments, a chimeric CAB-NP can include the N-terminal six amino acids of human ANP (SLRRSS; SEQ ID NO:14), the 17 amino acid ring structure and disulfide bond of human CNP (CFGLKLDRIGSMSGLGC; SEQ ID NO:11), and the C-terminal six amino acids of human BNP (KVLRRH; SEQ ID NO:19), and can have the amino acid sequence SLRRSSCFGLKLDRIGSMSGLGCKVLRRH (SEQ ID NO:20).

In some embodiments, a chimeric CBB-NP can include the N-terminal nine amino acids of human BNP (SPKMVQGSG; SEQ ID NO:21), the 17 amino acid ring structure and disulfide bond of human CNP (CFGLKLDRIGSMSGLGC; SEQ ID NO:11), and the C-terminal six amino acids of human BNP (KVLRRH; SEQ ID NO:19), and can have the amino acid sequence SPKMVQGSGCFGLKLDRIGSMSGLGCKVLRRH (SEQ ID NO:22).

In some embodiments, a chimeric CDD-NP can include the N-terminal six amino acids of DNP (EVKYDP; SEQ ID NO:23), the 17 amino acid ring structure and disulfide bond of human CNP (CFGLKLDRIGSMSGLGC; SEQ ID NO:11), and the C-terminal 15 amino acids of DNP (PSLRDPRPNAPSTSA; SEQ ID NO:7), and can have the amino acid sequence EVKYDPCFGLKLDRIGSMSGLGCPSLRDPRPNAPSTSA (SEQ ID NO:24).

In some embodiments, a chimeric CUB-NP can include the N-terminal 10 amino acids of human URO (TAPRSLRRSS; SEQ ID NO:10), the 17 amino acid ring structure and disulfide bond of human CNP (CFGLKLDRIGSMSGLGC; SEQ ID NO:11), and the C-terminal six amino acids of human BNP (KVLRRH; SEQ ID NO:19), and can have the amino acid sequence TAPRSLRRSSCFGLKLDRIGSMSGLGCKVLRRH (SEQ ID NO:25).

In some embodiments, a chimeric ABC-NP1 can include amino acids 11 to 15 of human ANP (RMDRI; SEQ ID NO:26) at its amino terminus, followed by the amino acid sequence of human CNP (GLSKGCFGLKLDRIGSMSGLGC; SEQ ID NO:3), and the C-terminal six amino acids of human BNP (KVLRRH; SEQ ID NO:19), and can have the amino acid sequence RMDRIGLSKGCFGLKLDRIGSMSGLGCKVLRRH (SEQ ID NO:27).

In some cases, a NP can include a variant (e.g., a substitution, addition, or deletion) at one or more positions (e.g., one, two, three, four, five, six, seven, eight, nine, or ten positions) with respect to any of SEQ ID NOs:1 to 27. For example, a chimeric ABC-NP can include amino acids 11 to 15 of human ANP (RMDRI; SEQ ID NO:26) at its amino terminus, followed by the amino acid sequence of human CNP with the exception that the amino acid residues at positions 15, 16, and 17 are changed to Arg, Glu, and Ala (GLSKGCFGLKLDRIREASGLGC; SEQ ID NO:28), followed by the C-terminal six amino acids of human BNP (KVLRRH; SEQ ID NO:19), and can have the amino acid RMDRIGLSKGCFGLKLDRIREASGLGCKVLRRH (SEQ ID NO:29). A chimeric BC-NP2 can include the amino acid sequence of human CNP with the exception that the amino acid residues at positions 15, 16, and 17 are changed to Arg, Glu, and Ala (GLSKGCFGLKLDRIREASGLGC; SEQ ID NO:28), followed by the C-terminal six amino acids of human BNP (KVLRRH; SEQ ID NO:19), and can have the amino acid sequence GLSKGCFGLKLDRIREASGLGCKVLRRH (SEQ ID NO:30).

Amino acid substitutions can be made, in some cases, by selecting substitutions that do not differ significantly in their effect on maintaining (a) the structure of the peptide backbone in the area of the substitution, (b) the charge or hydrophobicity of the molecule at the target site, or (c) the bulk of the side chain. For example, naturally occurring residues can be divided into groups based on side-chain properties: (1) hydrophobic amino acids (norleucine, methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Non-limiting examples of useful substitutions include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenyalanine, glycine for proline, threonine for serine, serine for threonine, tyrosine for tryptophan, phenylalanine for tyrosine, and/or leucine for valine.

Non-limiting examples of variant NPs can include the following:

(SEQ ID NO: 31)
TLRRSSCFGGRMDRIGAQSGLGCNSFRY
(SEQ ID NO: 32)
SIRRSSCFGGRMDRIGAQSGLGCNSFRY
(SEQ ID NO: 33)
SLKRSSCFGGRMDRIGAQSGLGCNSFRY
(SEQ ID NO: 34)
SLRKSSCFGGRMDRIGAQSGLGCNSFRY
(SEQ ID NO: 35)
SLRRSSCFGGRMDRIGAQSGLGCNTFRY
(SEQ ID NO: 36)
SLRRSSCFGGRMDRIGAQSGLGCNSLRY
(SEQ ID NO: 37)
SLRRSSCFGGRMDRIGAQSGLGCNSFKY
(SEQ ID NO: 38)
SLRRSSCFGGRMDRIGAQSGLGCNSFRF
(SEQ ID NO: 39)
TPKMVQGSGCFGRKMDRISSSSGLGCKVLRRH
(SEQ ID NO: 40)
SGKMVQGSGCFGRKMDRISSSSGLGCKVLRRH
(SEQ ID NO: 41)
SPRMVQGSGCFGRKMDRISSSSGLGCKVLRRH
(SEQ ID NO: 42)
SPKLVQGSGCFGRKMDRISSSSGLGCKVLRRH
(SEQ ID NO: 43)
SPKMVQGSGCFGRKMDRISSSSGLGCKVIRRH
(SEQ ID NO: 44)
SPKMVQGSGCFGRKMDRISSSSGLGCKVLKRH
(SEQ ID NO: 45)
SPKMVQGSGCFGRKMDRISSSSGLGCKVLRKH
(SEQ ID NO: 46)
SPKMVQGSGCFGRKMDRISSSSGLGCKVLRRR
(SEQ ID NO: 47)
PLSKGCFGLKLDRIGSMSGLGC
(SEQ ID NO: 48)
GISKGCFGLKLDRIGSMSGLGC
(SEQ ID NO: 49)
GLTKGCFGLKLDRIGSMSGLGC
(SEQ ID NO: 50)
GLSRGCFGLKLDRIGSMSGLGC
(SEQ ID NO: 51)
GLSKGCFGLKLDRIGSMSPLGC
(SEQ ID NO: 52)
GLSKGCFGLKLDRIGSMSGIGC
(SEQ ID NO: 53)
GLSKGCFGLKLDRIGSMSGLPC
(SEQ ID NO: 54)
GLSKGCFGLKLDRIGSMSGLGS
(SEQ ID NO: 55)
TPKMVQGSGCFGRKMDRISSSSGLGCPSLRDPRPNAPSTSA
(SEQ ID NO: 56)
SGKMVQGSGCFGRKMDRISSSSGLGCPSLRDPRPNAPSTSA
(SEQ ID NO: 57)
SPRMVQGSGCFGRKMDRISSSSGLGCPSLRDPRPNAPSTSA
(SEQ ID NO: 58)
SPKLVQGSGCFGRKMDRISSSSGLGCPSLRDPRPNAPSTSA
(SEQ ID NO: 59)
SPKMVQGSGCFGRKMDRISSSSGLGCPSLRDPRPNAPTTSA
(SEQ ID NO: 60)
SPKMVQGSGCFGRKMDRISSSSGLGCPSLRDPRPNAPSSSA
(SEQ ID NO: 61)
SPKMVQGSGCFGRKMDRISSSSGLGCPSLRDPRPNAPSTTA
(SEQ ID NO: 62)
SPKMVQGSGCFGRKMDRISSSSGLGCPSLRDPRPNAPSTSV
(SEQ ID NO: 63)
PLSKGCFGLKLDRIGSMSGLGCPSLRDPRPNAPSTSA
(SEQ ID NO: 64)
GISKGCFGLKLDRIGSMSGLGCPSLRDPRPNAPSTSA
(SEQ ID NO: 65)
GLTKGCFGLKLDRIGSMSGLGCPSLRDPRPNAPSTSA
(SEQ ID NO: 66)
GLSRGCFGLKLDRIGSMSGLGCPSLRDPRPNAPSTSA
(SEQ ID NO: 67)
GLSKGCFGLKLDRIGSMSGLGCPSLRDPRPNAPTTSA
(SEQ ID NO: 68)
GLSKGCFGLKLDRIGSMSGLGCPSLRDPRPNAPSSSA
(SEQ ID NO: 69)
GLSKGCFGLKLDRIGSMSGLGCPSLRDPRPNAPSTTA
(SEQ ID NO: 70)
GLSKGCFGLKLDRIGSMSGLGCPSLRDPRPNAPSTSV
(SEQ ID NO: 71)
SAPRSLRRSSCFGLKLDRIGSMSGLGCNSFRY
(SEQ ID NO: 72)
TVPRSLRRSSCFGLKLDRIGSMSGLGCNSFRY
(SEQ ID NO: 73)
TAGRSLRRSSCFGLKLDRIGSMSGLGCNSFRY
(SEQ ID NO: 74)
TAPKSLRRSSCFGLKLDRIGSMSGLGCNSFRY
(SEQ ID NO: 75)
TAPRSLRRSSCFGLKLDRIGSMSGLGCNTFRY
(SEQ ID NO: 76)
TAPRSLRRSSCFGLKLDRIGSMSGLGCNSLRY
(SEQ ID NO: 77)
TAPRSLRRSSCFGLKLDRIGSMSGLGCNSFKY
(SEQ ID NO: 78)
TAPRSLRRSSCFGLKLDRIGSMSGLGCNSFRF
(SEQ ID NO: 79)
TLRRSSCFGRKMDRISSSSGLGCNSFRY
(SEQ ID NO: 80)
SIRRSSCFGRKMDRISSSSGLGCNSFRY
(SEQ ID NO: 81)
SLKRSSCFGRKMDRISSSSGLGCNSFRY
(SEQ ID NO: 82)
SLRKSSCFGRKMDRISSSSGLGCNSFRY
(SEQ ID NO: 831)
SLRRSSCFGRKMDRISSSSGLGCNTFRY
(SEQ ID NO: 84)
SLRRSSCFGRKMDRISSSSGLGCNSLRY
(SEQ ID NO: 85)
SLRRSSCFGRKMDRISSSSGLGCNSFKY
(SEQ ID NO: 86)
SLRRSSCFGRKMDRISSSSGLGCNSFRF
(SEQ ID NO: 87)
SAPRSLRRSSCFGRKMDRISSSSGLGCNSFRY
(SEQ ID NO: 88)
TVPRSLRRSSCFGRKMDRISSSSGLGCNSFRY
(SEQ ID NO: 89)
TAGRSLRRSSCFGRKMDRISSSSGLGCNSFRY
(SEQ ID NO: 90)
TAPKSLRRSSCFGRKMDRISSSSGLGCNSFRY
(SEQ ID NO: 91)
TAPRSLRRSSCFGRKMDRISSSSGLGCNTFRY
(SEQ ID NO: 92)
TAPRSLRRSSCFGRKMDRISSSSGLGCNSLRY
(SEQ ID NO: 93)
TAPRSLRRSSCFGRKMDRISSSSGLGCNSFKY
(SEQ ID NO: 94)
TAPRSLRRSSCFGRKMDRISSSSGLGCNSFRF
(SEQ ID NO: 95)
TLRRSSCFGLKLDRIGSMSGLGCKVLRRH
(SEQ ID NO: 96)
SIRRSSCFGLKLDRIGSMSGLGCKVLRRH
(SEQ ID NO: 97)
SLKRSSCFGLKLDRIGSMSGLGCKVLRRH
(SEQ ID NO: 98)
SLRKSSCFGLKLDRIGSMSGLGCKVLRRH
(SEQ ID NO: 99)
SLRRSSCFGLKLDRIGSMSGLGCKVIRRH
(SEQ ID NO: 100)
SLRRSSCFGLKLDRIGSMSGLGCKVLKRH
(SEQ ID NO: 101)
SLRRSSCFGLKLDRIGSMSGLGCKVLRKH
(SEQ ID NO: 102)
SLRRSSCFGLKLDRIGSMSGLGCKVLRRR
(SEQ ID NO: 103)
TPKMVQGSGCFGLKLDRIGSMSGLGCKVLRRH
(SEQ ID NO: 104)
SGKMVQGSGCFGLKLDRIGSMSGLGCKVLRRH
(SEQ ID NO: 105)
SPRMVQGSGCFGLKLDRIGSMSGLGCKVLRRH
(SEQ ID NO: 106)
SPKLVQGSGCFGLKLDRIGSMSGLGCKVLRRH
(SEQ ID NO: 107)
SPKMVQGSGCFGLKLDRIGSMSGLGCKVIRRH
(SEQ ID NO: 108)
SPKMVQGSGCFGLKLDRIGSMSGLGCKVLKRH
(SEQ ID NO: 109)
SPKMVQGSGCFGLKLDRIGSMSGLGCKVLRKH
(SEQ ID NO: 110)
SPKMVQGSGCFGLKLDRIGSMSGLGCKVLRRR
(SEQ ID NO: 111)
DVKYDPCFGLKLDRIGSMSGLGCPSLRDPRPNAPSTSA
(SEQ ID NO: 112)
ELKYDPCFGLKLDRIGSMSGLGCPSLRDPRPNAPSTSA
(SEQ ID NO: 113)
EVRYDPCFGLKLDRIGSMSGLGCPSLRDPRPNAPSTSA
(SEQ ID NO: 114)
EVKFDPCFGLKLDRIGSMSGLGCPSLRDPRPNAPSTSA
(SEQ ID NO: 115)
EVKYDPCFGLKLDRIGSMSGLGCPSLRDPRPNAPTTSA
(SEQ ID NO: 116)
EVKYDPCFGLKLDRIGSMSGLGCPSLRDPRPNAPSSSA
(SEQ ID NO: 117)
EVKYDPCFGLKLDRIGSMSGLGCPSLRDPRPNAPSTTA
(SEQ ID NO: 118)
EVKYDPCFGLKLDRIGSMSGLGCPSLRDPRPNAPSTSV
(SEQ ID NO: 119)
SAPRSLRRSSCFGLKLDRIGSMSGLGCKVLRRH
(SEQ ID NO: 120)
TVPRSLRRSSCFGLKLDRIGSMSGLGCKVLRRH
(SEQ ID NO: 121)
TAGRSLRRSSCFGLKLDRIGSMSGLGCKVLRRH
(SEQ ID NO: 122)
TAPKSLRRSSCFGLKLDRIGSMSGLGCKVLRRH
(SEQ ID NO: 123)
TAPRSLRRSSCFGLKLDRIGSMSGLGCKVIRRH
(SEQ ID NO: 124)
TAPRSLRRSSCFGLKLDRIGSMSGLGCKVLKRH
(SEQ ID NO: 125)
TAPRSLRRSSCFGLKLDRIGSMSGLGCKVLRKH
(SEQ ID NO: 126)
TAPRSLRRSSCFGLKLDRIGSMSGLGCKVLRRR
(SEQ ID NO: 127)
KMDRIGLSKGCFGLKLDRIGSMSGLGCKVLRRH
(SEQ ID NO: 128)
RLDRIGLSKGCFGLKLDRIGSMSGLGCKVLRRH
(SEQ ID NO: 129)
RMERIGLSKGCFGLKLDRIGSMSGLGCKVLRRH
(SEQ ID NO: 130)
RMDKIGLSKGCFGLKLDRIGSMSGLGCKVLRRH
(SEQ ID NO: 131)
RMDRIGLSKGCFGLKLDRIGSMSGLGCKVIRRH
(SEQ ID NO: 132)
RMDRIGLSKGCFGLKLDRIGSMSGLGCKVLKRH
(SEQ ID NO: 133)
RMDRIGLSKGCFGLKLDRIGSMSGLGCKVLRKH
(SEQ ID NO: 134)
RMDRIGLSKGCFGLKLDRIGSMSGLGCKVLRRR
(SEQ ID NO: 135)
KMDRIGLSKGCFGLKLDRIREASGLGCKVLRRH
(SEQ ID NO: 136)
RLDRIGLSKGCFGLKLDRIREASGLGCKVLRRH
(SEQ ID NO: 137)
RMERIGLSKGCFGLKLDRIREASGLGCKVLRRH
(SEQ ID NO: 138)
RMDKIGLSKGCFGLKLDRIREASGLGCKVLRRH
(SEQ ID NO: 139)
RMDRIGLSKGCFGLKLDRIREASGLGCKVIRRH
(SEQ ID NO: 140)
RMDRIGLSKGCFGLKLDRIREASGLGCKVLKRH
(SEQ ID NO: 141)
RMDRIGLSKGCFGLKLDRIREASGLGCKVLRKH
(SEQ ID NO: 142)
RMDRIGLSKGCFGLKLDRIREASGLGCKVLRRR
(SEQ ID NO: 143)
PLSKGCFGLKLDRIREASGLGCKVLRRH
(SEQ ID NO: 144)
GISKGCFGLKLDRIREASGLGCKVLRRH
(SEQ ID NO: 145)
GLTKGCFGLKLDRIREASGLGCKVLRRH
(SEQ ID NO: 146)
GLSRGCFGLKLDRIREASGLGCKVLRRH
(SEQ ID NO: 147)
GLSKGCFGLKLDRIREASGLGCKVIRRH
(SEQ ID NO: 148)
GLSKGCFGLKLDRIREASGLGCKVLKRH
(SEQ ID NO: 149)
GLSKGCFGLKLDRIREASGLGCKVLRKH
(SEQ ID NO: 150)
GLSKGCFGLKLDRIREASGLGCKVLRRR

Further examples of conservative substitutions that can be made at any position within the polypeptides provided herein are set forth in Table 1.

TABLE 1
Examples of conservative amino acid substitutions

| Original | | Preferred |
|---|---|---|
| Residue | Exemplary substitutions | substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn | Asn |
| Glu | Asp | Asp |
| Gly | Pro | Pro |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, Gln, Asn | Arg |
| Met | Leu, Phe, Ile | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleucine | Leu |

In some embodiments, a NP can include one or more non-conservative substitutions. Non-conservative substitutions typically entail exchanging a member of one of the classes described above for a member of another class. Such production can be desirable to provide large quantities or alternative embodiments of such compounds. Whether an amino acid change results in a functional polypeptide can readily be determined by assaying the specific activity of the polypeptide variant.

A natriuretic polypeptide provided herein can have any appropriate sequence. For example, a natriuretic polypeptide can include the sequences set forth in SEQ ID NOs:6 and 7. In some cases, a natriuretic polypeptide provided herein can contain an amino acid sequence that aligns to (a) the sequence set forth in SEQ ID NO:6 with five or less (e.g., five or less, four or less, three or less, two or less, one, or zero) amino acid additions, deletions, substitutions, or combinations thereof, and (b) the sequence set forth in SEQ ID NO:7 with three or less (e.g., three or less, two or less, one, or zero) amino acid additions, deletions, substitutions, or combinations thereof. For example, a polypeptide provided herein can contain the sequence set forth in SEQ ID NO:8, with the exception that the first serine residue or the last alanine residue of SEQ ID NO:8 is deleted or replaced with a different amino acid residue. Other examples of natriuretic polypeptides that can be used as described herein are set forth in WO 2010/078325.

A natriuretic polypeptide provided herein can have any appropriate length. For example, a natriuretic polypeptide provided herein can be between 17 and 65 (e.g., between 18 and 40, between 22 and 44, between 25 and 45, between 26 and 44, between 27 and 43, between 28 and 42, between 29 and 41, between 30 and 40, between 31 and 39, between 23 and 35, between 25 and 30, or between 30 and 35) amino acid residues in length. It will be appreciated that a polypeptide with a length of 17 or 65 amino acid residues is a polypeptide with a length between 17 and 65 amino acid residues.

Polypeptides such as variant NPs having conservative and/or non-conservative substitutions (e.g., with respect to any of SEQ ID NOs:1 to 30) can be screened for biological activity using any suitable assays, including those described elsewhere (see, e.g., WO 2010/078325). For example, the activity of a NP as described herein can be evaluated in vivo by, for example, testing its effects on factors such as pulmonary capillary wedge pressure, right atrial pressure, mean arterial pressure, urinary sodium excretion, urine flow, proximal and distal fractional sodium reabsorption, plasma renin activity, plasma cGMP levels, urinary cGMP excretion, net renal generation of cGMP, glomerular filtration rate, and left ventricular mass in animals. In some cases, such parameters can be evaluated after induced MI (e.g., MI induced by coronary artery ligation). In some cases, a polypeptide provided herein can be evaluated in vivo by, for example, testing its effects on kidney cysts and/or kidney weight as described herein.

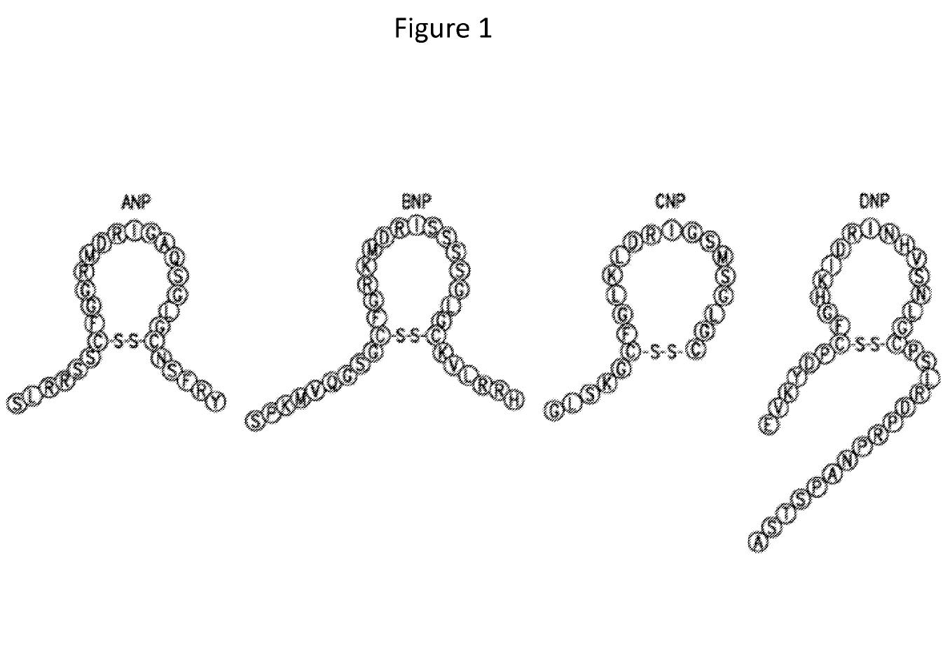

In some embodiments, the NPs provided herein can be cyclic due to disulfide bonds between cysteine residues (see, e.g., the ANP, BNP, CNP, and DNP structures depicted in FIG. 1). In some embodiments, a sulfhydryl group on a cysteine residue can be replaced with an alternative group (e.g., —CH₂CH₂—). To replace a sulfhydryl group with a —CH₂— group, for example, a cysteine residue can be replaced by alpha-aminobutyric acid. Such cyclic analog polypeptides can be generated, for example, in accordance with the methodology of Lebl and Hruby (Tetrahedron Lett., 25:2067 (1984)), or by employing the procedure disclosed in U.S. Pat. No. 4,161,521.

In addition, ester or amide bridges can be formed by reacting the OH of serine or threonine with the carboxyl group of aspartic acid or glutamic acid to yield a bridge having the structure —CH₂CO₂CH₂—. Similarly, an amide can be obtained by reacting the side chain of lysine with aspartic acid or glutamic acid to yield a bridge having the structure —CH₂C(O)NH(CH)₄—. Methods for synthesis of these bridges are described elsewhere (see, e.g., Schiller et al., Biochem. Biophy. Res. Comm., 127:558 (1985), and Schiller et al., Int. J. Peptide Protein Res., 25:171 (1985)). Other bridge-forming amino acid residues and reactions are provided in, for example, U.S. Pat. No. 4,935,492.

In some embodiments, a NP can comprise an amino acid sequence as set forth in SEQ ID NOs:1, 2, 3, 4, 5, 8, 9, 13, 16, 17, 18, 20, 22, 24, 25, 27, 29, or 30, but with a particular number of amino acid substitutions. For example, a NP can have the amino acid sequence of any one of SEQ ID NOs:1, 2, 3, 4, 5, 8, 9, 13, 16, 17, 18, 20, 22, 24, 25, 27, 29, or 30, but with one, two, three, four, or five amino acid substitutions. Examples of such amino acid sequences include, without limitation, those set forth in SEQ ID NOs:31-150.

Isolated polypeptides can be produced using any suitable methods, including solid phase synthesis, and can be generated using manual techniques or automated techniques (e.g., using an Applied BioSystems (Foster City, Calif.) Peptide Synthesizer or a Biosearch Inc. (San Rafael, Calif.) automatic peptide synthesizer. Disulfide bonds between cysteine residues can be introduced by mild oxidation of the linear polypeptides using KCN as taught, e.g., in U.S. Pat. No. 4,757,048. NPs also can be produced recombinantly, as described herein.

In some cases, a polypeptide provided herein can be a substantially pure polypeptide. As used herein, the term “substantially pure” with reference to a polypeptide means that the polypeptide is substantially free of other polypeptides, lipids, carbohydrates, and nucleic acid with which it is naturally associated. Thus, a substantially pure polypeptide is any polypeptide that is removed from its natural environment and is at least 60 percent pure or is any chemically synthesized polypeptide. A substantially pure polypeptide can be at least about 60, 65, 70, 75, 80, 85, 90, 95, or 99 percent pure. Typically, a substantially pure polypeptide will yield a single major band on a non-reducing polyacrylamide gel.

Salts of carboxyl groups of polypeptides can be prepared by contacting the polypeptide with one or more equivalents of a desired base such as, for example, a metallic hydroxide base (e.g., sodium hydroxide), a metal carbonate or bicarbonate base (e.g., sodium carbonate or sodium bicarbonate), or an amine base (e.g., triethylamine, triethanolamine, and the like). Acid addition salts of polypeptides can be prepared by contacting the polypeptide with one or more equivalents of an inorganic or organic acid (e.g., hydrochloric acid).

Esters of carboxyl groups of polypeptides can be prepared using any suitable method for converting a carboxylic acid or precursor to an ester. For example, one method for preparing esters of a polypeptide, when using the Merrifield synthesis technique, is to cleave the completed polypeptide from the resin in the presence of the desired alcohol under either basic or acidic conditions, depending upon the resin. The C-terminal end of the polypeptide then can be directly esterified when freed from the resin, without isolation of the free acid.

Amides of polypeptides can be prepared using techniques for converting a carboxylic acid group or precursor to an amide. One method for amide formation at the C-terminal carboxyl group includes cleaving the polypeptide from a solid support with an appropriate amine, or cleaving in the presence of an alcohol, yielding an ester, followed by aminolysis with the desired amine

N-acyl derivatives of an amino group of a polypeptide can be prepared by using an N-acyl protected amino acid for the final condensation, or by acylating a protected or unprotected peptide. O-acyl derivatives can be prepared for example, by acylation of a free hydroxy peptide or peptide resin. Either acylation may be carried out using standard acylating reagents such as acyl halides, anhydrides, or acyl imidazoles. Both N- and O-acylation may be carried out together, if desired.

In some embodiments, a NP can be modified by linkage to a polymer such as polyethylene glycol (PEG), or by fusion to another polypeptide such as albumin, for example. For example, one or more PEG moieties can be conjugated to a NP via lysine residues. Linkage to PEG or another suitable polymer, or fusion to albumin or another suitable polypeptide can result in a modified NP having an increased half life as compared to an unmodified NP. Without being bound by a particular mechanism, an increased serum half life can result from reduced proteolytic degradation, immune recognition, or cell scavanging of the modified NP. Methods for modifying a polypeptide by linkage to PEG (also referred to as “PEGylation”) or other polymers include those set forth in U.S. Pat. No. 6,884,780; Cataliotti et al. (Trends Cardiovasc. Med., 17:10-14 (2007)); Veronese and Mero (BioDrugs, 22:315-329 (2008)); Miller et al., (Bioconjugate Chem., 17:267-274 (2006)); and Veronese and Pasut (Drug Discov. Today, 10:1451-1458 (2005)). Methods for modifying a polypeptide by fusion to albumin include those set forth in U.S. Patent Publication No. 20040086976, and Wang et al. (Pharm. Res., 21:2105-2111 (2004)).

A NP as provided herein can function through the one or more of the guanylyl cyclase receptors through which the native NPs function. For example, in some embodiments, a NP that binds to and functions through the NPR-A receptors through which ANP and BNP function can be used as described herein to reduce the generation of kidney cysts, to reduce the number of kidney cysts, to reduce the size of kidney cysts, and/or to reduce the weight of a mammal's kidneys in mammals with polycystic kidney disease.

As described herein, mammals having polycystic kidney disease and identified as being in need of a reduction in kidney cyst generation, the number of kidney cysts, the size of kidney cysts, and/or the kidney weight can be administered a natriuretic polypeptide or nucleic acid designed to express a natriuretic polypeptide under conditions wherein the mammal is exposed to the natriuretic polypeptides over an extended duration (e.g., greater than one, two, three, four, five, six, or more months) to reduce the generation of kidney cysts, to reduce the number of kidney cysts, to reduce the size of kidney cysts, and/or to reduce the weight of a mammal's kidneys.

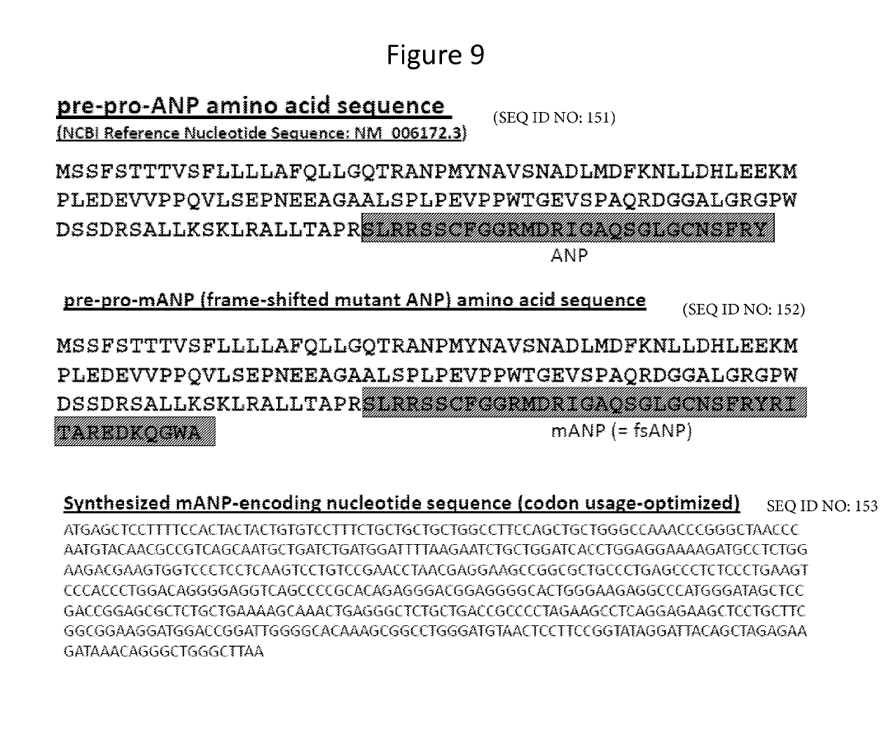

As described herein, nucleic acid vectors can be configured to include a nucleic acid sequence that encodes a natriuretic polypeptide. Examples of nucleic acid vectors that can be designed to express a nucleic acid sequence encoding a natriuretic polypeptide include, without limitation, AAV9 vectors, AAV2 vectors, AAV8 vectors, AAV vectors with natural AAV serotype capsid sequences, AAV vectors with designed AAV capsid sequences, adenoviral vectors, lentiviral vectors, transposases, episomal vectors, RNA-based viral vectors (e.g., measles, Sendai, and Borna disease viruses), and vectors designed for site-directed gene editing. Examples of natriuretic polypeptides that can be expressed using a nucleic acid vector (e.g., an AAV9 vector), without limitation, those natriuretic polypeptides described herein such as ANP (e.g., human ANP), BNP (e.g., human BNP), CNP (e.g., human CNP), CDNP, DNP, mANP, and ASBNP. The amino acid sequence for ANP, pre-pro-ANP, mANP, and pre-pro-mANP can be as set forth in FIG. 9. In some cases, the nucleic acid sequence encoding a natriuretic polypeptide can be codon optimized. For example, a codon optimized nucleic acid sequence designed to encode pre-pro-mANP as set forth in FIG. 9 can be used to make an AAV9 or AAV2 vector provided herein.

In some cases, a nucleic acid vector (e.g., an AAV9 vector) can be configured to include two or more different nucleic acid sequences that encode natriuretic polypeptides. For example, an AAV9 vector can be configured to include a nucleic acid sequence that encodes human BNP and a nucleic acid sequence that encodes CDNP.

In some cases, the one or more natriuretic polypeptides to be expressed using a nucleic acid vector (e.g., an AAV9 vector) can include the N-terminal region of a natural natriuretic polypeptide that includes non-active components of an active natriuretic polypeptide such as a signal peptide sequence and other sequences that can be involved in polypeptide processing, folding, and stabilization. Examples of such N-terminal regions include, without limitation, those set forth in SEQ ID NO: 1, 4, or 5 of WO2013/103896. In some cases, one or more of the following sequences can be used as an N-terminal region of a natriuretic polypeptide to be expressed using a nucleic acid vector (e.g., an AAV9 vector): BNP signal peptide+NT-proBNP, CNP signal peptide+NT-proCNP, and ANP signal peptide+NT-proANP. Examples of amino acid sequences encoding a natriuretic polypeptide that includes such an N-terminal region include, without limitation, those amino acid sequences set forth in SEQ ID NO: 3, 7, 8, or 13 of WO2013/103896.

A nucleic acid sequence (e.g., a nucleic acid sequence optimized for human codon usage) encoding a natriuretic polypeptide described herein can be inserted into any appropriate nucleic acid vector. For example, a nucleic acid sequence encoding a human CDNP can be inserted into an AAV9 vector having a nucleic acid sequence as set forth in GenBank® Accession No. AY530557 (GI No. 46487760), JA400113.1 (GI No. 346220229), JA232063 (GI No. 330731135), JA231827 (GI No. 330729561), or JA062576 (GI No. 328343515). In some cases, an AAV9 vector can have the sequence as described elsewhere. See, e.g., WO2003/052052, U.S. Patent Application Publication No. 20110236353, EP2345731, EP2292780, EP2292779, or EP2298926.

In some cases, a promoter sequence can be operably linked to a nucleic acid sequence that encodes a natriuretic polypeptide (e.g., BNP, pre-proBNP, CDNP, B-CDNP, or C-CDNP) to drive expression of the natriuretic polypeptide. Examples of such promoter sequences include, without limitation, CMV, EF1alpha, BNP, CNP, ANP, MYH6, and MYH7 promoters. In some cases, a promoter sequence that is active under conditions of elevated blood pressure with minimal, or no, activity under conditions of normal or low blood pressure can be operably linked to a nucleic acid sequence that encodes a natriuretic polypeptide (e.g., BNP, pre-proBNP, CDNP, B-CDNP, or C-CDNP) to drive expression of the natriuretic polypeptide under conditions of elevated blood pressure. Examples of such blood pressure sensitive promoter sequences include, without limitation, BNP and ANP promoters.

Any appropriate method can be used to insert nucleic acid (e.g., nucleic acid encoding a natriuretic polypeptide) into a nucleic acid vector (e.g., an AAV9 vector). For example, standard molecule biology techniques such as restriction enzyme cutting, ligations, and homologous recombination can be used to insert nucleic acid into an AAV9 vector. Any appropriate method can be used to identify a nucleic acid vector (e.g., an AAV9 vector) containing a nucleic acid molecule that encodes a natriuretic polypeptide. Such methods include, without limitation, PCR and nucleic acid hybridization techniques such as Northern and Southern analysis. In some cases, immunohistochemistry and biochemical techniques can be used to determine if a nucleic acid vector (e.g., an AAV9 vector) contains a particular nucleic acid molecule by detecting the expression of a polypeptide encoded by that particular nucleic acid molecule.

An AAV9 vector provided herein can be produced in human cell lines, such as 293T cells, or other types of cells such as insect cells, which can be concentrated typically by at least 100-fold, or even by as much as 5,000- to 10,000-fold, through ultracentrifugation. A viral titer typically is assayed by measuring the viral vector copy numbers in concentrated/purified vector preparations.

As described herein, a nucleic acid vector (e.g., an AAV9 vector) can be administered to a patient (e.g., human patient) by, for example, direct injection into a group of cells or intravenous delivery to cells. A nucleic acid vector (e.g., an AAV9 vector) can be administered to a patient in a biologically compatible solution or a pharmaceutically acceptable delivery vehicle such as saline, by administration either directly into a group of cells or systemically (e.g., intravenously). Suitable pharmaceutical formulations depend in part upon the use and the route of entry. Such forms should not prevent the composition or formulation from reaching a target cell (i.e., a cell to which the virus is desired to be delivered to) or from exerting its effect. For example, pharmacological compositions injected into the blood stream should be soluble.

While dosages administered will vary from patient to patient, an effective dose can be determined by setting as a lower limit the concentration of virus proven to be safe and escalating to higher doses of up to 10¹⁴ vector genome copies (vg)/kg, while monitoring for a response (e.g., a reduction in kidney cyst formation) along with the presence of any deleterious side effects. Escalating dose studies can be used to obtain a desired effect for a given viral treatment.

A nucleic acid vector (e.g., an AAV9 vector) can be delivered in a dose ranging from, for example, about 10³ vg/kg to about 10¹⁴ vg/kg. A therapeutically effective dose can be provided in repeated doses. Repeat dosing is appropriate in cases in which observations of clinical symptoms or monitoring assays indicate that the degree of viral activity (e.g., natriuretic polypeptide expression) is declining. Repeat doses can be administered by the same route as initially used or by another route. A therapeutically effective dose can be delivered in several discrete doses (e.g., days, weeks, months, or years apart).

A nucleic acid vector (e.g., an AAV9 vector) can be directly administered to cells (e.g., kidney, heart, liver, thymous, pancreas, brain, or skeletal muscle cells). For example, a virus can be injected directly into kidney tissue. In some cases, ultrasound guidance can be used in such a method. In some cases, a nucleic acid vector (e.g., an AAV9 vector) can be delivered systemically. For example, systemic delivery can be achieved intravenously via injection. The course of therapy with a nucleic acid vector (e.g., an AAV9 vector) can be monitored by evaluating changes in clinical symptoms.

In some cases, a natriuretic polypeptide or a nucleic acid encoding a natriuretic polypeptide can be incorporated into a composition for administration to a mammal (e.g., a human known to have polycystic kidney disease). Such composition can be given once or more daily, weekly, monthly, or even less often, or can be administered continuously for a period of time (e.g., hours, days, or weeks). For example, a natriuretic polypeptide can be administered continuously as an infusion or via a pump delivery system for at least about three months to nine months at a dose of about 0.01 ng NP/kg/minute to about 0.5 ng NP/kg/minute. In some cases, a single administration of a nucleic acid vector (e.g., an AAV9 vector or an AAV2 vector) designed to express a natriuretic polypeptide can be used to deliver natriuretic polypeptide for at least about three or more months (e.g., four or more months, five or more months, six or more months, seven or more months, eight or more months, nine or more months, ten or more months, eleven or more months, or twelve or more months).

The natriuretic polypeptides and nucleic acids encoding a natriuretic polypeptide can be admixed, encapsulated, conjugated or otherwise associated with other molecules, molecular structures, or mixtures of compounds such as, for example, liposomes, receptor or cell targeted molecules, or oral, topical or other formulations for assisting in uptake, distribution, and/or absorption.

In some embodiments, a composition containing a natriuretic polypeptide or a nucleic acid encoding a natriuretic polypeptide can include a pharmaceutically acceptable carrier. Pharmaceutically acceptable carriers include, for example, pharmaceutically acceptable solvents, suspending agents, or any other pharmacologically inert vehicles for delivering polypeptides or nucleic acids to a subject. Pharmaceutically acceptable carriers can be liquid or solid, and can be selected with the planned manner of administration in mind so as to provide for the desired bulk, consistency, and other pertinent transport and chemical properties, when combined with one or more therapeutic compounds and any other components of a given pharmaceutical composition. Typical pharmaceutically acceptable carriers include, without limitation, saline solutions, binding agents (e.g., polyvinylpyrrolidone or hydroxypropyl methylcellulose), fillers (e.g., lactose or dextrose and other sugars, gelatin, or calcium sulfate), lubricants (e.g., starch, polyethylene glycol, or sodium acetate), disintegrates (e.g., starch or sodium starch glycolate), and wetting agents (e.g., sodium lauryl sulfate).

A composition containing a natriuretic polypeptide or a nucleic acid encoding a natriuretic polypeptide can be administered by a number of methods, depending upon whether local or systemic treatment is desired. Administration can be, for example, parenteral (e.g., by subcutaneous, intrathecal, intraventricular, intramuscular, or intraperitoneal injection, or by intravenous (i.v.) drip); oral; topical (e.g., transdermal, sublingual, ophthalmic, or intranasal); or pulmonary (e.g., by inhalation or insufflation of powders or aerosols), or can occur by a combination of such methods. Administration can be rapid (e.g., by injection) or can occur over a period of time (e.g., by slow infusion or administration of slow release formulations). In some cases, a natriuretic polypeptide can be formulated as a sustained release dosage form. For example, a natriuretic polypeptide such as BNP can be formulated into a controlled release formulation. In some cases, coatings, envelopes, or protective matrices can be formulated to contain one or more natriuretic polypeptides. In some cases, a natriuretic polypeptide can incorporated into a polymeric substances, liposomes, microemulsions, microparticles, nanoparticles, or waxes.

The invention will be further described in the following example, which does not limit the scope of the invention described in the claims.

### EXAMPLE

### Reducing Kidney Size and Kidney Cystogenesis in Mammals with Polycystic Kidney Disease

The following was performed to demonstrate the anti-cystogenic effects of a particulate guanylyl cyclase A (GC-A) agonist, BNP, in a rat model of polycystic kidney disease.

### Sustained BNP Over-Expression Significantly Reduced the Kidney Size and Cystogenesis in Female PKD Rats Independently of the Blood Pressure Effect

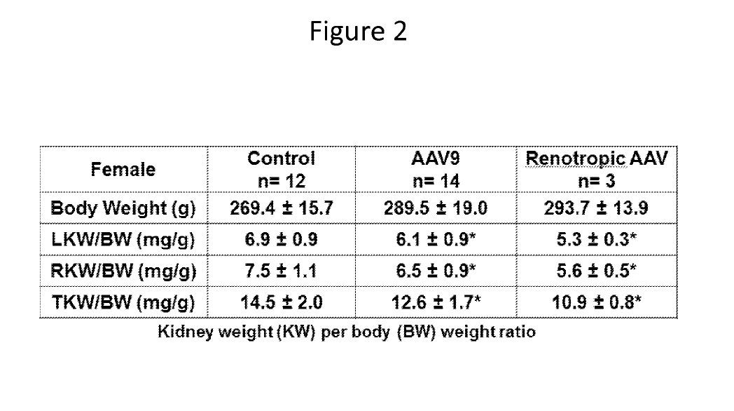

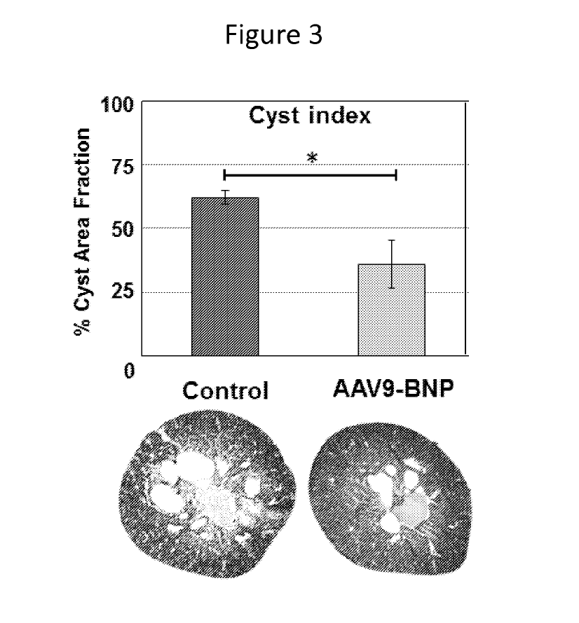

PCK rats were used as a rat model of PKD, which are characterized by progressive renal and hepatic cyst formation. For sustained BNP treatment, adeno-associated virus (AAV) serotypes 9- and 2-based vectors (AAV9 and AAV2 vectors were used to express BNP. AAV9 vector (10¹³ genome copies (gc)/kg) were delivered to induce BNP over-expression in newborn rats. Three months of BNP overexpression by the AAV9 vectors significantly reduced kidney size over 13% (FIG. 2) and cystogenesis (FIG. 3) in female PKD rats. AAV2-BNP vector treatment (n=3) also reduced kidney size (FIG. 2).

### High Dose AAV9-BNP Vector Treatment Strongly Suppressed Both Renal and Hepatic Cyst Formation

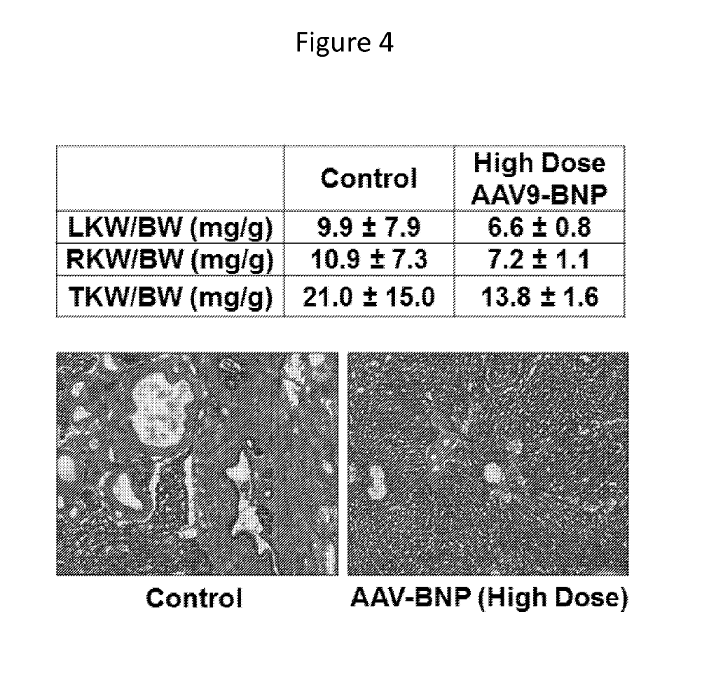

Female PCK rats, treated by AAV9-BNP vectors at a high dose (10¹⁴ gc/kg) for 4.5 months, also demonstrated cyst inhibition and more than 30% reduction in kidney size (FIG. 4, upper panel). Notably, chronic high dose BNP treatment also strongly blocked hepatic cystogenesis in female PKD rats (FIG. 4, lower panel).

These results demonstrate that sustained BNP treatment reduced the kidney size and renal and hepatic cystogenesis. Thus, guanylyl cyclase agonists (e.g., GC-A agonists such as BNP) can provide potent anti-cystogenic effects for the treatment of PKD cystogenesis.

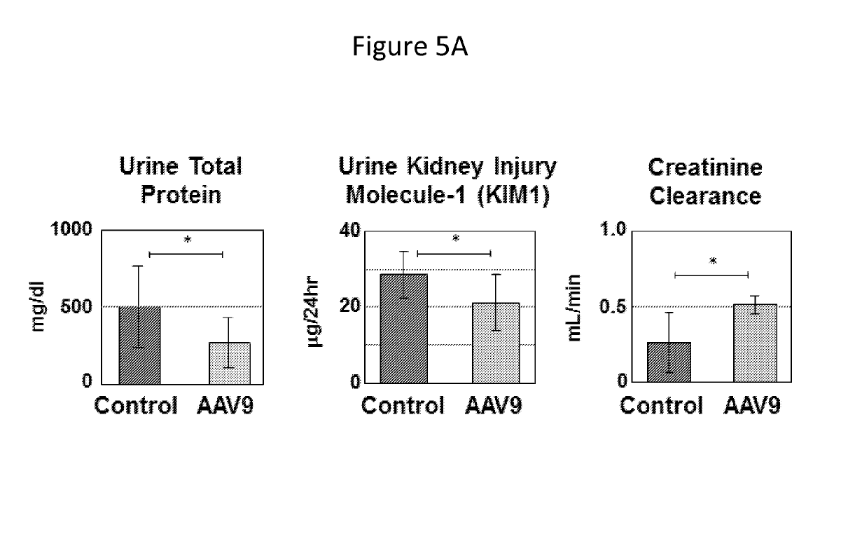

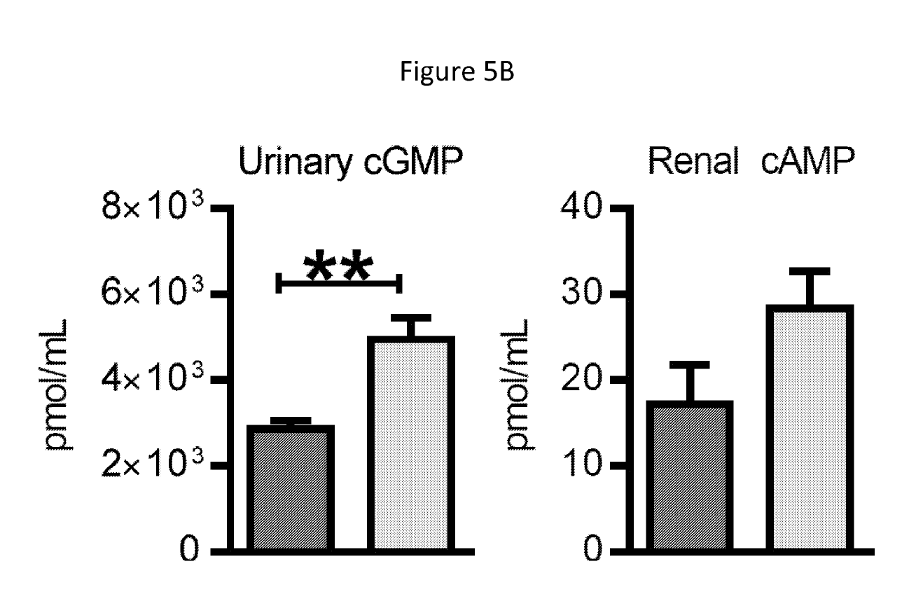

It is noted that these effects were accompanied by the concomitant improvement in cardiovascular and renal function, which also was observed in the BNP-treated PKD rats. For example, AAV9-BNP vector treatment for 3 months also significantly reduced proteinuria and urinary excretion of KIM1 (a marker of tubular injury, FIG. 5A) and NGAL (an epithelial injury marker), while preserving creatinine clearance (FIG. 5A). Cyclic guanosine monophosphate (cGMP) was increased in AAV9-BNP vector treated PCK (FIG. 5B). cAMP levels were not significantly distinct between treatment groups, though appeared elevated with BNP therapy (FIG. 5B).

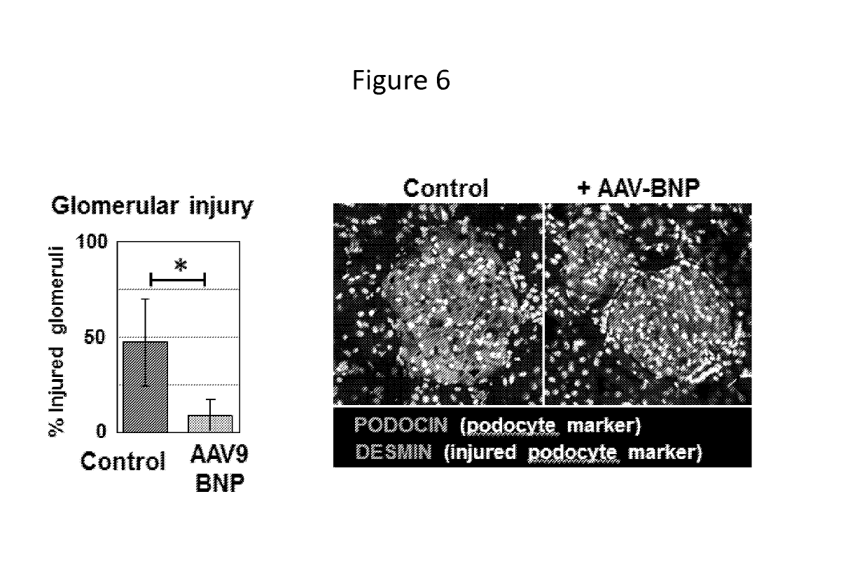

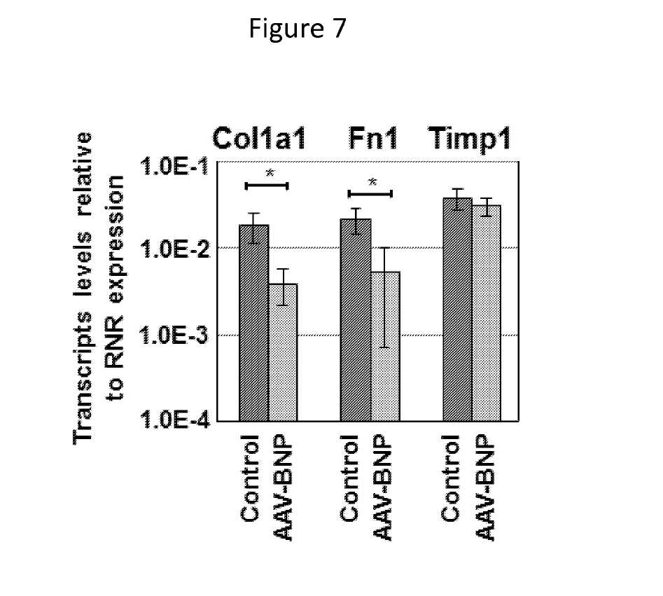

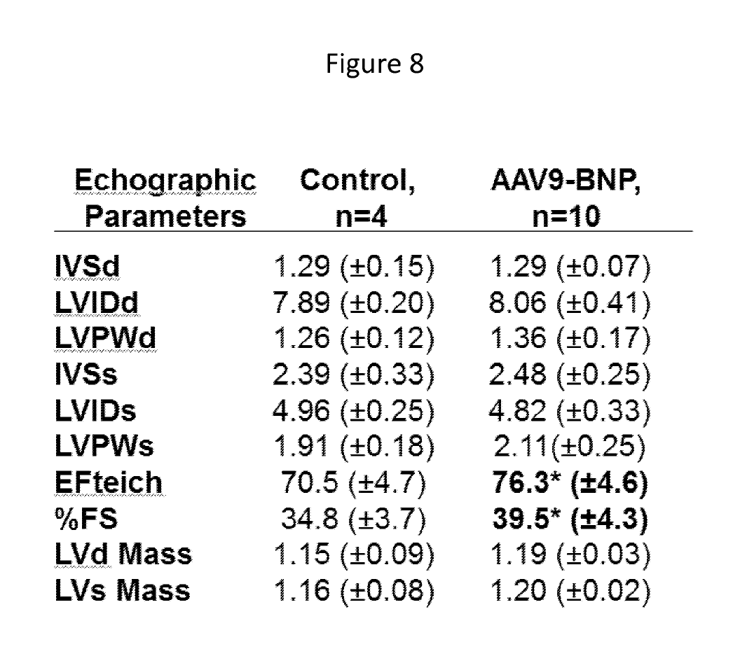

These effects were coupled with a significant reduction in the number of injured glomeruli characterized as sclerotic or having thickened basement membrane (FIG. 6, left panel). Confocal imaging revealed prominent induction of glomerular DESMIN expression, a marker specific to expanded mesangial cells and podocyte injury in untreated cystic kidneys (FIG. 6, right panel). Quantitative RT-PCR analysis also revealed significant suppression of profibrotic genes, including collagen I and fibronectin (FIG. 7). Long-term BNP treatment also was associated with improved cardiac function (FIG. 8) in PKD.

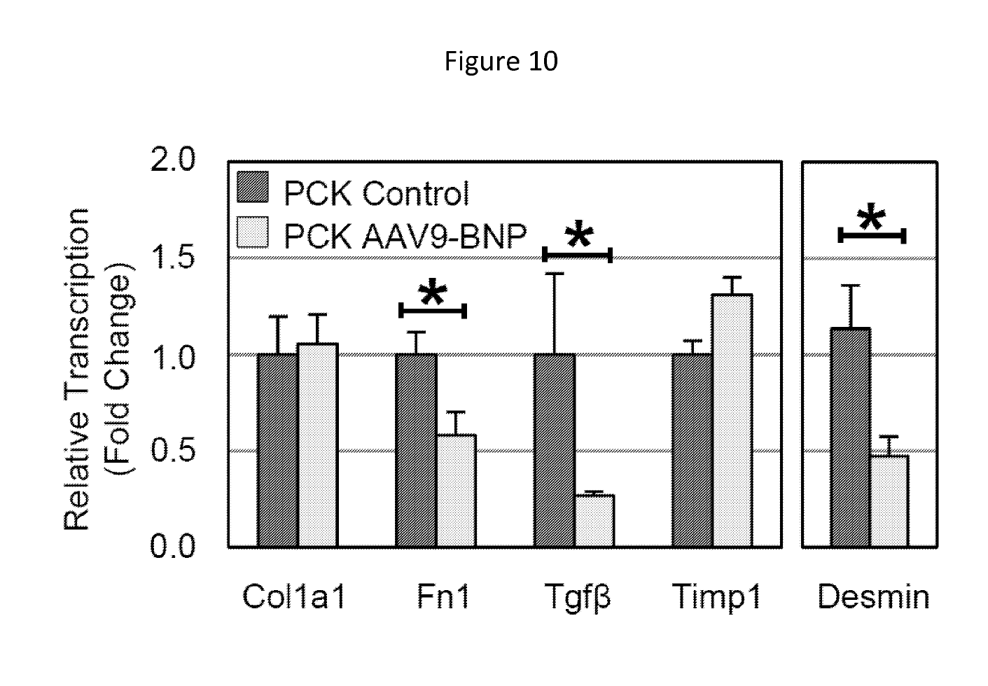

Systemic and long term AAV9-BNP vector administration resulted in a significant reduction in profibrosis gene expression of Fn1, Tgfβ, and desmin (FIG. 10).

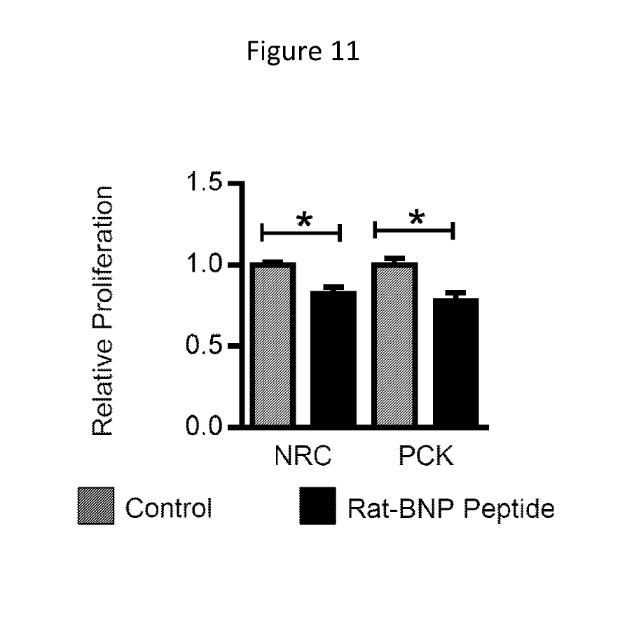

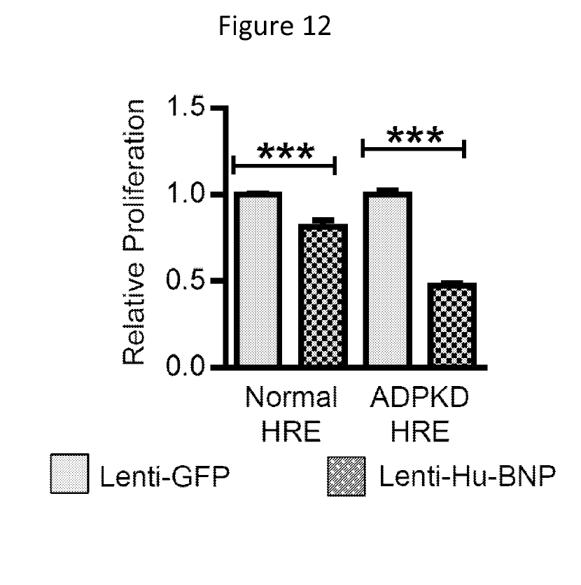

BNP peptide treatment resulted in a reduction in both normal and cystic cholangiocyte proliferation, with a more aggressive anti-proliferative effect in murine cystic epithelial cells (FIG. 11). Healthy and cystic renal epithelial proliferation was significantly depressed after human-BNP lentiviral transduction, relative to control GFP-lenti cell lines (FIG. 12).

Long-term NP (e.g., BNP) therapy can be achieved through gene delivery using various vectors (e.g., AAV vectors with natural or designed AAV capsid proteins) or can be achieved through the repeated administration of NP in the form of polypeptides.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal diagnosed with polycystic kidney disease, wherein said method comprises:
(a) identifying said mammal as being in need of a reduction in kidney cysts, and
(b) administering a natriuretic polypeptide or a nucleic acid encoding a natriuretic polypeptide to said mammal, wherein the number or size of kidney cysts within said mammal are reduced.

2. The method of claim 1, wherein said mammal is a cat, dog, or human.

3. The method of claim 1, wherein said natriuretic polypeptide is BNP.

4. The method of claim 1, wherein said method comprises administering a natriuretic polypeptide to said mammal at least three times a week for at least three months.

5. The method of claim 1, wherein said method comprises administering said nucleic acid to said mammal.

6. The method of claim 5, wherein said nucleic acid is a viral vector.

7. The method of claim 5, wherein said nucleic acid is an AAV9 or AAV2 viral vector.

8. A method for treating a mammal diagnosed with polycystic kidney disease, wherein said method comprises:
(a) identifying said mammal as being in need of a reduction in kidney weight, and
(b) administering a natriuretic polypeptide or a nucleic acid encoding a natriuretic polypeptide to said mammal, wherein the weight of a kidney within said mammal is reduced.

9. The method of claim 8, wherein said mammal is a cat, dog, or human.

10. The method of claim 8, wherein said natriuretic polypeptide is BNP.

11. The method of claim 8, wherein said method comprises administering a natriuretic polypeptide to said mammal at least three times a week for at least three months.

12. The method of claim 8, wherein said method comprises administering said nucleic acid to said mammal.

13. The method of claim 12, wherein said nucleic acid is a viral vector.

14. The method of claim 12, wherein said nucleic acid is an AAV9 or AAV2 viral vector.
